(19) United States
(12) Patent Application Publication
(10) Pub. No.: US 2021/0068444 A1
(43) Pub. Date: Mar. 11, 2021

(54) CANNABINOID COMPOSITIONS WITH IMPROVED ORGANOLEPTIC AND THERAPEUTIC PROPERTIES, METHOD OF PRODUCTION, AND USE THEREOF

(71) Applicant: CALIFORNIA AMBER INC., Los Gatos, CA (US)

(72) Inventors: Ramon Alarcon, Los Gatos, CA (US); Steven Edward Brown, Oak Ridge, NC (US)

(21) Appl. No.: 17/017,650

(22) Filed: Sep. 10, 2020

Related U.S. Application Data
(60) Provisional application No. 62/898,000, filed on Sep. 10, 2019.

(51) Int. Cl. A24B 15/16; A24F 40/20; A61K 9/00; A24F 40/10; A61K 31/05; A61K 31/352

(52) U.S. Cl. CPC A24B 15/16; A24F 40/20; A61K 31/352; A24F 40/10; A61K 31/05; A61K 9/0078

(57) ABSTRACT

Provided herein are compositions comprising cannabinoids, terpenoids, and other flavonoids. Also provided herein are methods for producing compositions comprising cannabinoids, terpenoids, and other flavonoids at industrial scale. In certain embodiments, the compositions provided herein possess desirable organoleptic properties and therapeutic effects when ingested or topically applied. In certain embodiments, the compositions of the present disclosure have are useful in inhalable, ingestible, or topical products to relieve and treat various acute or chronic illnesses.

### CROSS REFERENCE TO RELATED APPLICATIONS

This application claims the benefit of U.S. Provisional Application No. 62/898,000, filed Sep. 10, 2019, which is incorporated by reference in the disclosure of this application.

### BACKGROUND

A need exists in the art for cannabinoid-based compositions with widely appealing organoleptic properties. Such properties include, but are not limited to, odor, flavor, and color.

### BRIEF SUMMARY OF THE DISCLOSURE

In an aspect, provided herein is a composition, comprising: a cannabis composition, comprising at least one cannabinoid and at least one flavonoid.

In some embodiments, the at least one flavonoid is a bioflavonoid, an isoflavonoid, a neoflavonoid, or combinations thereof. In some embodiments, the at least one flavonoid is a natural flavonoid, a synthetic flavonoid, or a combination thereof. In some embodiments, the at least one flavonoid is a natural flavonoid. In some embodiments, the composition comprises flavonoids at a concentration of from about 0.1% to about 15% by weight.

In some embodiments, the at least one cannabinoid is a natural cannabinoid, a synthetic cannabinoid, or a combination thereof. In some embodiments, the at least one cannabinoid is a natural cannabinoid. In some embodiments, the composition comprises cannabinoids at a concentration of from about 0.1% to about 30% by weight.

In some embodiments, the composition further comprises at least one terpenoid and/or terpene. In some embodiments, the at least one terpenoid and/or terpene is a natural terpenoid and/or terpene.

In some embodiments, the composition is derived or extracted from a plant material.

In some embodiments, the composition is fully derived or extracted or partially derived or extracted from a plant substrate. In some embodiments, the plant substrate is plant material. In some embodiments, the plant material is plant flowers, plant leaves, plant stems, plant stalks, or combinations thereof. In some embodiments, the plant material is a cannabis plant, a hemp plant, or a combination thereof. In some embodiments, the cannabis plant material comprises at least one species from the cannabaceae family

In some embodiments, the composition is derived by exposing the plant substrate to heat under a gas atmosphere.

In some embodiments, the plant substrate is heated to a temperature of from about 100° C. to about 500° C. In some embodiments, the plant substrate is heated to a temperature of from about 150° C. to about 350° C.

In some embodiments, the gas environment comprises at least one of nitrogen gas, argon gas, carbon dioxide gas, helium gas, or oxygen gas. In some embodiments, the gas environment comprises up to about 21% oxygen by weight. In some embodiments, the gas environment comprises up to about 15% oxygen by weight. In some embodiments, the gas environment comprises up to about 10% oxygen by weight. In some embodiments, the gas environment is essentially free of oxygen. In some embodiments, a reaction vessel is provided. In some embodiments, the reaction vessel is a closed system vessel. In some embodiments, the gas environment is at atmospheric pressure, below atmospheric pressure, or above atmospheric pressure. In some embodiments, the gas environment comprises a gas flow. In some embodiments, the gas environment comprises a continuous gas flow. In some embodiments, the non-stationary gas flow is a gas stream.

In some embodiments, the at least one cannabinoid comprises a cannabigerol (CBG), a cannabichromene (CBC), a cannabidiol (CBD), tetrahydrocannabinol (THC), cannabinol (CBN), cannabielsoin (CBE), iso-tetrahydrocannabinol (iso-THC), cannabicyclol (CBL), cannabicitran (CBT), CBG-type cannabinoid a CBC-type cannabinoid, a CBD-type cannabinoid, a THC-type cannabinoid, a CBN-type cannabinoid, a CBE-type cannabinoid, an iso-THC-type cannabinoid, a CBL-type cannabinoid, a CBT-type cannabinoid, a CBG-type cannabinoid, or combinations thereof.

In some embodiments, the at least one cannabinoid comprises THC, CBD, CBC, CBN, CBG, CBL, a THC-type cannabinoid, a CBD-type cannabinoid, a CBC-type cannabinoid, a CBN-type cannabinoid, a CBG-type cannabinoid, a CBL-type cannabinoid, or combinations thereof.

In some embodiments, the THC-type cannabinoid is tetrahydrocannabinolic acid (THCA), tetrahydrocannabivarin (THCV), Δ⁹-THC, Δ⁹-THC, 8-hydroxy-Δ⁹-tetrahydrocannabinol, or combinations thereof.

In some embodiments, the THC-type cannabinoid is Δ⁸-THC.

In some embodiments, the at least one cannabinoid comprises a CBC-type cannabinoid, cannabidiolic acid (CBGA), cannabichromene, a CBC-type cannabinoid, CBD, or combinations thereof. In some embodiments, the cannabinoid is CBD.

In some embodiments, the composition further comprises a fatty acid or ester thereof. In some embodiments, the fatty acid or ester thereof comprises a short- or medium-chain fatty acid or ester thereof. In some embodiments, the fatty acid ester is a C1-C6 ester. In some embodiments, the fatty acid esterisamethylester. In some embodiments, the composition further comprises: 2,2-dimethyloxetane; pentanal; dimethyl disulfide; pyridine; 1-acetylcyclohexene; acetamide; 3-furaldehyde; p-mentha-1,3,8-triene; p-mentha-1,5,8-triene; 6-methyl-5-hepten-2-one; 2,4-dimethyl-2,6-heptadien-1-ol; benzaldehyde; butyrolactone; (1R,5S)-2-methylene-6,6-dimethylbicyclo[311]heptane; p-(1-propenyl)-toluene; p-cymenene; acetophenone; caryophyllene; humulene; 2-isopropenyl-4a,8-dimethyl-1,2,3,4,4a,5,6,7-octahydronaphthalene; alpha-guaiene; 1,2-benzenediol, [2-(2-methylpropoxycarbonyloxy)phenyl] 4-butylbenzoate; (8aR)-8a-methyl-4-methylidene-6-propan-2-ylidene-2,3,4a,5,7,8-hexahydro-1H-naphthalene; neophytadiene; 1,4-dimethyl-7-prop-1-en-2-yl-1,2,3,3a,4,5,6,7-octahydroazulene; cannabichromene; (6aR,8R,10aR)-6,6,9-trimethyl-3-pentyl-6a,7,8,10a-tetrahydrobenzo[c]chromene-1,8-diol; 1H-tetrazole; allyl acetate; (1R)-2,6,6-trimethylbicyclo[311]hept-2-ene; (1R,5S)-2-methylene-6,6-dimethylbicyclo[311]heptane; 2,3-Dihydroxypropyl acetate; (1aS,3aR,8bR,8cR)-1,1,3a-trimethyl-6-pentyl-1a,2,3,3a,8b,8c-hexahydro-1H-4-oxabenzo[f]cyclobuta[cd]inden-8-ol; dronabinol; 7-isopropyl-4a,8a-dimethyloctahydro-1(2H)-naphthalenone; n-propyl 9,12-octadecadienoate; methyl 9,12,15-octadecatrienoate; 6,9 octadecadienoic acid methyl ester; ethyl linolenate; butyl 9,12-octadecadienoate; 9,12-octadecadienoyl chloride (z,z); methyl 9-cis, 11 trans-octadecadienoate; 2-mono-linolein; methyl (11E,14E)-octadeca-11,14-dienoate; ethyl linolenate; methyl heptadeca-8,11,14-trienoate; methyl (9Z,12Z,15Z)-2-hydroxyoctadeca-9,12,15-trienoate; methyl (7E,10E,13E)-hexadeca-7,10,13-trienoate; butyl (9Z,12Z,15Z)-9,12,15-octadecatrienoate; methyl (5Z,8Z,11Z,14Z)-icosa-5,8,11,14-tetraenoate; n-propyl 9,12,15-octadecatrienoate, or combinations thereof.

In some embodiments, the composition further comprises at least one carrier, excipient, or additive. In some embodiments, the at least one carrier, excipient, or additive is selected from diluents, antiadherents, binders, coatings, disintegrants, surfactants, dissolving agents, solubilizing agents, bioadhesive agents, polysaccharides, polymers, copolymers, bioavailability enhancing agents, thin film-type excipient, mucoadhesive agents, acidifying agents, probiotic agents, protective agents, antioxidants, dispersing agents, flavors, color additives, fragrance, lubricants, glidants, sorbents, preservatives, sweeteners, or combinations thereof. In some embodiments, the at least one carrier, excipient, or additive is at least one solvent or solvent system. In some embodiments, the at least one solvent or solvent system comprises an organic solvent, an inorganic solvent, or a combination thereof. In some embodiments, the at least one solvent or solvent system comprises a polar solvent, a non-polar solvent, or a combination thereof. In some embodiments, the composition is miscible with the solvent or solvent system.

In some embodiments, the composition is immiscible with the solvent or solvent system. In some embodiments, the at least one solvent or solvent system comprises an oil. In some embodiments, the oil is MCT oil, olive oil, canola oil, hemp oil, or combinations thereof. In some embodiments, the at least one solvent is or solvent system comprises water, alcohol, a hydrocarbon, an ether, or combinations thereof. In some embodiments, the alcohol is ethanol, propylene glycol, glycerol, or combinations thereof. In some embodiments, the alcohol is ethanol. In some embodiments, the composition comprises ethanol from about 0.01% to about 60% by weight ethanol.

In some embodiments, the hydrocarbon is a fluorinated hydrocarbon. In some embodiments, the fluorinated hydrocarbon is tetrafluoroethylene, propane, butane, or a combination thereof. In some embodiments, the ether is polyethylene glycol (PEG). In some embodiments, the ether is polyethylene glycol 400 (PEG 400). Certain embodiments further comprise a co-solvent.

In some embodiments, the composition further comprises at least one preservative. In some embodiments, the composition is in the form of an oil, a liquid, a solid, or a gas. In some embodiments, the liquid is a solution, a suspension, or an emulsion. In some embodiments, the composition is in the form of a paste, a cream, a gel, a liniment or balm, an aerosol, a lotion, an ointment, drops, a concentrate, a skin patch, an oral or nasal spray, a film, a food or beverage additive, an edible food product, a tablet, a capsule, a fast dissolving tablet (FDT), an effervescent tablet, a syrup, an elixir, a cartridge, or a suppository.

In some embodiments, the composition is formulated for oral delivery, topical delivery, enteral delivery, parenteral delivery, intranasal delivery, sublingual delivery, buccal delivery, inhalation delivery. In some embodiments, the inhalation delivery is inhalation by mouth, inhalation by nose, or a combination thereof.

In some embodiments, the inhalation delivery is by inhaler, nebulizer, vaporizer, aerosolizer, or a smoking device. In some embodiments, the smoking device is a cigarette, cigar, pipe, or an electronic smoking device

In some embodiments, the smoking device is a cigarette or cigar comprising tobacco, hemp cannabis, herbs, spices, or combinations thereof. In some embodiments, the device is an electronic smoking device. In some embodiments, the vaporizer is an open system, a semi-open system, or closed system vaporizer.

In some embodiments, the composition has a peak with a retention time of about 6.6 min in a GC-MS chromatogram. In some embodiments, the composition has a peak with a retention time of about 6.1 min in a GC-MS chromatogram. In some embodiments, the composition has a peak with a retention time of about 6.2 min in a GC-MS chromatogram. In some embodiments, the composition has a peak with a retention time of about 6.3 min in a GC-MS chromatogram. In some embodiments, the composition has a peak with a retention time of about 6.4 min in a GC-MS chromatogram. In some embodiments, the composition has a peak with a retention time of about 6.5 min in a GC-MS chromatogram. In some embodiments, the composition has a peak with a retention time of about 6.7 min in a GC-MS chromatogram. In some embodiments, the composition has a peak with a retention time of about 6.8 min in a GC-MS chromatogram. In some embodiments, the composition has a peak with a retention time of about 6.9 min in a GC-MS chromatogram.

In some embodiments, the composition has at least one additional peak with a retention time of about 4.5, about 4.6, about 4.7, about 4.8, about 4.9, or about 7.15 minutes in a GC-MS chromatogram. In some embodiments, the composition has at least two additional peaks with a retention time of about 4.5, about 4.6, about 4.7, about 4.8, about 4.9, or about 7.15 minutes in a GC-MS chromatogram. In some embodiments, the composition has at least three additional peaks with a retention time of about 4.5, about 4.6, about 4.7, about 4.8, about 4.9, or about 7.15 minutes in a GC-MS chromatogram. In some embodiments, the composition has at least four additional peaks with a retention time of about 4.5, about 4.6, about 4.7, about 4.8, about 4.9, or about 7.15 minutes in a GC-MS chromatogram. In some embodiments, the composition is characterized by a GC-MS chromatogram of FIG. 28.

In another aspect, provided herein is a process for producing the compositions disclosed herein, comprising:

(i) providing an unprocessed plant feedstock in a gas environment;

(ii) heating the unprocessed plant feedstock in the gas environment to afford a processed plant product and at least one volatile compound; and

(iii) extracting the treated plant product of (ii) with a first solvent or solvent system thereby providing a first extract

In some embodiments, the plant material is heated to a temperature of from about 100° C. to about 500° C. In some embodiments, the plant material is heated to a temperature of from about 150° C. to about 350° C.

In some embodiments, the gas environment comprises at least one of nitrogen gas, argon gas, carbon dioxide gas, helium gas, or oxygen gas. In some embodiments, the gas environment comprises up to about 21% oxygen by weight. In some embodiments, the gas environment comprises up to about 15% oxygen by weight. In some embodiments, the gas environment comprises up to about 10% oxygen by weight. In some embodiments, the gas environment is essentially free of oxygen. In some embodiments, the gas environment is at atmospheric pressure, below atmospheric pressure, or above atmospheric pressure. In some embodiments, the gas environment comprises a gas flow. In some embodiments, the gas flow is a gas stream. In some embodiments, the gas flow is pressurized gas.

In some embodiments, the first solvent or solvent system comprises an organic solvent, an inorganic solvent, or a combination thereof. In some embodiments, the first solvent or solvent system comprises a polar solvent, a non-polar solvent, or a combination thereof. In some embodiments, the first solvent or solvent system comprises water, alcohol, a hydrocarbon, an ether, CO₂, or combinations thereof. In some embodiments, the alcohol is ethanol.

In some embodiments, the hydrocarbon is a fluorinated hydrocarbon.

In some embodiments, the fluorinated hydrocarbon is tetrafluoroethylene.

In some embodiments, the hydrocarbon is propane, butane, or a combination thereof. In some embodiments, the CO₂ is supercritical CO₂, sub-critical CO₂, or a combination thereof. Certain embodiments of the process described herein further comprise a first co-solvent. In some embodiments, the first solvent or solvent system is mono-phasic or bi-phasic. In some embodiments, the first extract is concentrated. In some embodiments, the first extract is concentrated by evaporation at atmospheric pressure, evaporation under vacuum, or a combination thereof. In some embodiments, the first extract is concentrated by application of heat. In some embodiments, the process further comprises combining the first extract with a first additive. In some embodiments, the first additive is ethanol, propylene glycol, glycerol, polyethylene glycol 400 (PEG 400), MCT oil, olive oil, canola oil, hemp oil, cannabis oil, or combinations thereof.

In some embodiments, the process further comprises capturing and extracting the at least one volatile compound with a second solvent or solvent system, an adsorbent substrate, or a combination thereof thereby providing a second extract.

In some embodiments, the second solvent or solvent system comprises an organic solvent, an inorganic solvent, or a combination thereof. In some embodiments, the second solvent or solvent system comprises a polar solvent, a non-polar solvent, or a combination thereof. In some embodiments, the second solvent or solvent system comprises water, alcohol, a hydrocarbon, an ether, CO₂, or combinations thereof. In some embodiments, the second solvent or solvent system comprises an oil. In some embodiments, the oil comprises an MCT oil, olive oil, canola oil, hemp oil, cannabis oil, or combinations thereof. In some embodiments, the alcohol is ethanol, propylene glycol, glycerol, or combinations thereof. In some embodiments, the hydrocarbon is a fluorinated hydrocarbon. In some embodiments, the fluorinated hydrocarbon is tetrafluoroethylene. In some embodiments, the hydrocarbon is propane, butane, or a combination thereof. In some embodiments, the ether is polyethylene glycol (PEG). In some embodiments, the ether is polyethylene glycol 400 (PEG 400). In some embodiments, the CO₂ is supercritical CO₂, sub-critical CO₂, or a combination thereof. In some embodiments of the process disclosed herein there is a second co-solvent. In some embodiments, the second solvent or solvent system is mono-phasic or bi-phasic. In some embodiments, the second extract is concentrated. In some embodiments, the second extract is concentrated by evaporation at atmospheric pressure, evaporation under vacuum, or a combination thereof. In some embodiments, the second extract is concentrated by application of heat. In some embodiments, the process comprises entraining the at least one volatile compound in a flowing gas stream. In some embodiments, the flowing gas stream comprises at least one of nitrogen gas, argon gas, carbon dioxide gas, helium gas, or oxygen gas

In some embodiments, the flowing gas stream comprises up to about 21% oxygen by weight. In some embodiments, the flowing gas stream comprises up to about 15% oxygen by weight. In some embodiments, the flowing gas stream comprises up to about 10% oxygen by weight. In some embodiments, the flowing gas stream is essentially free of oxygen.

In some embodiments, the process further comprises combining the second extract with a second additive. In some embodiments, the second additive is ethanol, propylene glycol, glycerol, polyethylene glycol 400 (PEG400), MCT oil, olive oil, canola oil, hemp oil, cannabis oil, or combinations thereof.

In some embodiments, the first extract is combined with the second extract. Certain embodiments of the processes described herein, further comprise combining the unprocessed plant feedstock of (i) with an additive. In some embodiments, the additive is a pH adjusting agent, a protein, a carbohydrate, or combinations thereof. In some embodiments, the pH adjusting agent is a bicarbonate, an alkali metal hydroxide, an amine, ammonia, or combinations thereof. In some embodiments, the bicarbonate is sodium bicarbonate. In some embodiments, about 0.5% to about 25% by weight of sodium bicarbonate is added to the unprocessed plant feedstock of (i). In some embodiments, about 2% to about 15% by weight of sodium bicarbonate is added to the unprocessed plant feedstock of (i). In some embodiments, the pH adjusting agent is a bicarbonate, an alkali metal hydroxide, an amine, ammonia, or combinations thereof. In some embodiments, the bicarbonate is sodium bicarbonate.

In an aspect, provided herein is a method of treating a disease or condition, comprising administering to a subject in need thereof a therapeutically effective amount of the composition of any one of the embodiments disclosed herein.

In some embodiments, the disease or condition is pain, insomnia, depression, Crohn's disease, multiple sclerosis, colitis, anxiety, post-traumatic stress disorder (PTSD), seizures, glaucoma, cancer, side effects from cancer treatment, bulimia, anorexia, obesity, a skin disorder, or nausea.

In an aspect, provided herein is a cigarette comprising the composition of any one of the embodiments disclosed herein. In some embodiments, the cigarette comprises tobacco, hemp cannabis, herbs, spices, or combinations thereof. In some embodiments, the cigarette is an electronic cigarette.

In another aspect, provided herein is a vaporizer cartridge comprising the composition of any one of the embodiments disclosed herein.

In another aspect, provided herein is a kit comprising the composition of any one of the embodiments disclosed herein in some embodiments, the kit further comprises instructions for use. In some embodiments, the kit further comprises a delivery device.

### INCORPORATION BY REFERENCE

All publications, patents, and patent applications mentioned in this specification are herein incorporated by reference to the same extent as if each individual publication, patent, or patent application was specifically and individually indicated to be incorporated by reference

### BRIEF DESCRIPTION OF THE DRAWINGS

The accompanying drawings, which are included to provide a further understanding of the disclosure, are incorporated in and constitute a part of this specification, illustrate embodiments of the disclosure and together with the detailed description serve to explain the principles of the disclosure. No attempt is made to show structural details of the disclosure in more detail than may be necessary for a fundamental understanding of the disclosure and the various ways in which it may be practiced.

FIG. 1 shows generalized decarboxylation reactions for THC and CBD.

FIG. 2 shows a generalized relationship of the heating time and temperature on THC content, illustrating that a maximum content exists for a given temperature and time at prolonged thermal exposure, THC content diminishes.

FIG. 3 shows a non-limiting example of terpene analysis of cannabis.

FIG. 4 shows a generalized process flow illustrating the primary functional components to enable the disclosure.

FIG. 5 shows a generalized diagram of process components to create the unique chemical compositions according to the disclosure

FIG. 6 shows a generalized process scheme to continuously produce compositions according to the disclosure.

FIG. 7 shows a generalized process diagram for a two-chamber operation useful for point of sale application.

FIG. 8 shows portions of a small-scale equipment setup used to produce compositions according to the disclosure.

FIG. 9 shows additional portions of a small-scale equipment setup used to product compositions according to the disclosure.

FIGS. 10A-10C show a table of experimental conditions to produce compositions according to the disclosed disclosure. The data contained in the table are non-limiting examples of process conditions to create compositions according to the disclosure

FIG. 11 shows a control composition made using cannabis flower without thermal process and a composition made according to the disclosure having superior organoleptic properties.

FIG. 12 illustrates that an optimal processing region exists to produce compositions with preferred chemical compositions that have desirable organoleptic characteristics.

FIG. 13 shows a control composition made using cannabis flower without thermal process and another composition made according to the disclosure having superior organoleptic properties.

FIG. 14 shows a spider diagram illustrating the organoleptic characteristics associated with the novel chemistry of the inhalable/ingestible composition according to the disclosure compared to compositions that are not made according to the disclosure, but represent current market place products. Circles and solid line are data associated with compositions made with extracts from non-thermally treated cannabis, which represents the control sample. Squares and dashed line are data associated with compositions made according to disclosure.

FIG. 15 shows a graph representing product consumability rated and based on the ability to inhale the composition over a prolonged period of 30 minutes to 2 hours, consumability also relating to the user not becoming impaired after consumption.

FIG. 16 shows a composition made according to the disclosure mixed with a composition not made according to the disclosure, the combined mixture having superior organoleptic properties to the composition not made according to the disclosure.

FIGS. 17A-17B show examples of a vaporizer article that is constructed according to an aspect of the disclosure.

FIG. 18 shows another example of a vaporizer article that is constructed according to an aspect of the disclosure.

FIG. 19 shows detailed elements of a vaporizer article that is constructed according to an aspect of the disclosure.

FIG. 20 shows an example of a cartridge that is constructed according to an aspect of the disclosure.

FIGS. 21A-21B show another example of a vaporizer article that is constructed according to an aspect of the disclosure.

FIG. 22 shows an example of a solid media vaporizer article that is constructed according an aspect of the disclosure.

FIGS. 23A-23C show an example of an ingestible delivery device that is constructed according to an aspect of the disclosure.

FIG. 24 shows an example of how composition information is displayed according to an aspect of the disclosure.

FIG. 25 shows an example of how composition information is displayed according to an aspect of the disclosure.

FIG. 26 shows an example process for treating substrate material with a composition made according to the disclosure.

FIG. 27 is a schematic representation of the processes described herein.

FIG. 28 shows overlaid GC-MS spectra of processed (Spectrum B) and unprocessed compositions (Spectrum A) created from equivalent starting materials and mass. The X-intercept indicates temperature of from 120 C to 300 C.

FIG. 29 is amass spectrum of processed extract minus unprocessed @ 6.58 minutes.

FIG. 30 is a mass spectrum of processed extract minus unprocessed @ 6.66 minutes.

FIG. 31 is a tabular representation of extract according to the process exemplary cannabinoid analysis determined by a method consistent with AOC method 2018.11.

FIG. 32 is a tabular representation of extract without the process exemplary cannabinoid analysis determined by a method consistent with AOC method 2018.11.

FIG. 33 is a table showing extract according to the process exemplary terpene analysis.

FIG. 34 shows extract without the process exemplary terpene analysis.

### DETAILED DESCRIPTION

Provided herein are, for example, compositions comprising cannabinoids and flavonoids, and derivatives thereof, methods of producing the compositions; and methods of use. In the processes for producing the compositions disclosed herein, a plant substrate is exposed to heat under a gas atmosphere. In some embodiments, the gas atmosphere is essentially free of oxygen. In some embodiments, the gas atmosphere comprises up to about 10% by volume of oxygen.

In some embodiments, the heat applied to the plant substrate is sufficient to generate chemical transformations of certain plant substrate components. In some embodiments, a plant product/extract afforded from a plant substrate exposed to heat under gaseous conditions has different physical properties and/or characteristics than the product/extract obtained from the plant substrate in the absence of heat exposure under gaseous conditions. In some embodiments, a plant product/extract afforded from a plant substrate exposed to heat has different physical properties and/or characteristics than the product/extract obtained from the plant substrate not exposed to heat.

In some embodiments, It also has different properties than product/extract obtained from the plant substrate exposed to heat, but not under our unique gaseous conditions

In some embodiments, a plant product/extract afforded from a plant substrate exposed to heat under gaseous conditions has a different chemical profile than the product/extract obtained from the plant substrate in the absence of heat exposure under gaseous conditions (e.g., comprises new components/compounds, changes in relative amounts of compounds/components as measured relative to a standard control sample.)

In some embodiments, changes in the characteristics and/or chemical makeup can lead to different physical properties of plant substrate products or extracts including, but not limited to solubility and/or homogeneity. In certain aspects, the processes described herein can be used to alter or modify organoleptic properties (e.g., altering flavor, aroma, taste) of a plant product/extract by changing the chemical profile and/or physical properties of a plant substrate. In certain aspects, the processes described herein can be used to alter or modify pharmaceutical properties (e.g., bioavailability, solubility) of a plant product/extract by changing the chemical profile and/or physical properties of a plant substrate.

In certain embodiments, the plant product is an intermediate. In certain embodiments, the plant product is a final product. In some embodiments, the plant substrate is plant material including but not limited to stalks, leaves, flowers, etc. In certain embodiments the plant is a cannabis plant, a hemp plant, or combinations thereof.

### I. Definitions

The abbreviations used herein have their conventional meaning within the chemical and biological arts.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

A “substituent group,” as used herein, means a group selected from the following moieties:
(A) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
   (i) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, heteroaryl, substituted with at least one substituent selected from:
      (a) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

Certain compounds disclosed herein possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The presently disclosed compounds include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope hereof.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope hereof.

The compounds of the present disclosure may also contain unnatural proportions of atomic isotopes (i.e., isotopic variants) at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the term “isomers” refers to compounds having the same number and kind of atoms, and hence the same molecular weight, but differing in respect to the structural arrangement or configuration of the atoms.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C1-C20 alkyl, or unsubstituted 2 to 20 membered heteroalkyl,” the group may contain one or more unsubstituted C1-C20 alkyls, and/or one or more unsubstituted 2 to 20 membered heteroalkyls.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts,” Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents. In embodiments, compounds of the present disclosure contain both basic and acidic functionalities that allow the compounds to be converted into either base or acid addition salts. The neutral forms of the compounds may be regenerated by contacting the salt with a base or acid and isolating the parent compound in a conventional manner. The parent form of the compounds differs from the various salt forms in certain physical properties, such as solubility in polar solvents, but, unless specifically indicated, the salts disclosed herein are equivalent to the parent form of the compound for the purposes of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of a compound to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway. In some embodiments contacting includes allowing a compound described herein to interact with another compound, solvent, gas, etc.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including MDS, AML, ALL, ATLL and CML), or multiple myeloma. As used herein, the term “cancer” refers to all types of cancer, neoplasm or malignant tumors found in mammals (e.g. humans), including leukemia, carcinomas and sarcomas.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatinifori carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA). In some embodiments, the autoimmune disease is not asthma.

As used herein, the term “inflammatory disease” refers to a disease or condition characterized by aberrant inflammation (e.g. an increased level of inflammation compared to a control such as a healthy person not suffering from a disease). Examples of inflammatory diseases include traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis.

The terms “treating” or “treatment” refer to any indicia of success in therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of a compound described herein. The administering step may consist of a single administration or may include a series of administrations. The length of the treatment period depends on a variety of factors, such as the severity of the condition, the age of the patient, the concentration of the compound, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and duration sufficient to treat the patient.

The terms “prevent,” “preventing,” and “prevention” refer to a decrease in the occurrence of disease symptoms in a patient. The preventing or prevention may be complete (no detectable symptoms) or partial, such that fewer symptoms are observed than would likely occur absent treatment. In embodiments, prevent refers to slowing the progression of the disease, disorder or condition or inhibiting progression thereof to a harmful or otherwise undesired state.

“Patient” or “subject in need thereof” refers to a living organism suffering from or prone to a disease or condition that can be treated by administration of a pharmaceutical composition as provided herein. In some embodiments, a patient is human.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). Therapeutically effective amount can be ascertained by measuring relevant physiological effects, and it can be adjusted in connection with the dosing regimen and diagnostic analysis of the subject's condition, and the like. By way of example, measurement of the serum level of a compound of Formula (I) or a hydrate, solvate, tautomer, or pharmaceutically acceptable salt thereof (or, e.g., a metabolite thereof) at a particular time post-administration may be indicative of whether a therapeutically effective amount has been administered.

For any compound described herein, therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan. Adjusting the dose to achieve maximal therapeutic window efficacy or toxicity in humans based on the methods described above and other methods is well within the capabilities of the ordinarily skilled artisan.

The term “therapeutically effective amount,” as used herein, refers to that amount of therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., chemotherapeutic agent). The compound of the disclosure can be administered alone or can be coadministered to the patient. Co-administration is meant to include simultaneous or sequential administration of the compound individually or in combination (more than one compound or agent). Thus, the preparations can also be combined, when desired, with other active substances (e.g., to reduce metabolic degradation). The compositions of the present disclosure can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present disclosure can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present disclosure can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present disclosure can also be delivered as nanoparticles.

By “co-administer” it is meant that a composition described herein is administered at the same time, just prior to, or just after the administration of one or more additional therapies. The compounds of the disclosure can be administered alone or can be coadministered to the patient. Coadministration is meant to include simultaneous or sequential administration of the compounds individually or in combination (more than one compound). The compositions of the present disclosure can be delivered transdermally, by a topical route, or formulated as applicator sticks, solutions, suspensions, emulsions, gels, creams, ointments, pastes, jellies, paints, powders, and aerosols.

For any compound described herein, therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present disclosure should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating cancer (e.g., colon cancer), cardiovascular disease, metabolic disease, immune or inflammatory disease or disorder.

In some embodiments, co-administration includes administering one active agent within 0.5, 1, 2, 4, 6, 8, 10, 12, 16, 20, 24 hours, 2 days, 4 days, 1 week or 1 month of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another. In some embodiments, the compounds described herein may be combined with treatments for cancer or infections (e.g., fungal infections, bacterial infections, viral infections, etc.)

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples). In some embodiments, the control is unprocessed substrate, feedstock, and/or plant material.

The phrase “in a sufficient amount to effect a change” means that there is a detectable difference between a level of an indicator measured before (e.g., a baseline level) and after administration of a particular therapy. Indicators include any objective parameter (e.g., serum concentration) or subjective parameter (e.g., a subject's feeling of well-being).

“Substantially pure” indicates that a component makes up greater than about 50% of the total content of the composition, and typically greater than about 60% of the total polypeptide content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition (percentage in a weight per weight basis).

“Substantially free” indicates that a component is totally absent or partially absent. In some embodiments, substantially free refers to a component that is undetectable by conventional means in the chemical sciences (e.g., NMR, HPLC, GC-MS, IR, elemental analysis, etc.). In other embodiments, substantially free refers to a component that under or less than a certain threshold value.

“Vaporizer” as used herein contemplates all devices used for nebulizing and/or aerosolizing a liquid composition. In some embodiments, the vaporizer is an open-system vaporizer, i.e., reusable as the e-liquid material to be vaporized can be replenished once it has run out, coils heating elements can be replaced, etc. Non-limiting examples of open-system vaporizers include vape pens. In some embodiments, the vaporizer is closed-system vaporizer, i.e., uses permanently connected or integrated refillable or non-refillable pods or cartridge that is not refillables. In some embodiments, the vaporizer is a semi open-system vaporizer, i.e., can be used with blank non-refillable cartridges or, pre-filled cartridge.

Some vaporizers can aerosolize solids and semi-solids for example waxes. Some vaporizers can vaporize dry herbs as well. Through heat, they liberate volatile components of the dry media. These components are entrained in the air stream to create and aerosol.

“Gas environment” as used herein refers to process environments or reaction conditions that are not under vacuum. In some embodiments, the gas or gas system is continuously moving through the reaction chamber/vessel during the process, e.g., a gas stream or a gas flow. In other embodiments, the gas or gas system is not continuously moving (i.e., is not passing through or sweeping the reaction chamber/vessel) in the processes described herein. In some embodiments, process conditions without continuous gas or gas system flow comprise gas or gas systems that are replenished via a source of flowing/streaming gas. In some embodiments, process conditions continuous gas or gas system flow comprise gas or gas systems that are replenished via a source of flowing/streaming gas. In some embodiments, gas gradients are used, i.e., changing concentration of gases during a single process.

Unless otherwise specified, the percentage concentrations disclosed herein refer to weight %. The terms and abbreviations “wt %,” “weight %,” “w/w,” and “percent by weight are used synonymously.

The use of cannabinoid containing products has gained more wide spread use and recognition for their therapeutic benefits and ability to improve overall quality of life for those who suffer from a wide range of maladies and disorders. This is relevant given the discovery of the endocannabinoid system and its role in biological function in humans. THC- and CBD-based compositions have shown therapeutic efficacy in the treatment of various medical conditions or disorders and/or the sequelae thereof. As non-limiting examples, these compositions can be ingested by inhalation of a mist or vapor, smoking plant-based material, oral tinctures or lozenges, edible foods, or other forms such as drinkable compositions as prescribed or over the counter medications. Furthermore, these compositions can be incorporated into, for example, topical creams, ointments, cosmetics, or other formulations. To that end, there is a need in the market place for these products to be user friendly and tolerable to reap the full benefit of their therapeutic value. In response to that need, sophisticated, expensive processes have been developed to isolate specific cannabinoids such as CBD and/or THC from other cannabinoids and terpenoids. These isolated cannabinoids can be used as is or further incorporated into other compositions.

In contrast, therapeutic values of the 113 known cannabinoids and combinations thereof are generally recognized for their potential and have found use in extracted products that are known as broad spectrum products. In general, these products also contain other cannabis chemistries such as terpenes and flavonoids, which are contained within the plant and may be captured by extraction processing technologies known in the art. Apart from THC and CBD, and to a lesser extent, CBN, CBG and CBC, isolation of the individual 113 cannabinoids from the terpenes and flavonoids is difficult and as such not commercially feasible. As a result, full spectrum cannabinoid containing products and some limited cannabinoid containing products, contain sufficient terpenes and flavonoids to impart a distinct taste and odor, known as organoleptic characteristics. These components give the distinctive odor and flavor to cannabis and hemp. In addition, many different species of cannabis and hemp are bred and different agricultural practices optimized to achieve desired organoleptic characteristics and properties for specialized market segments. To that end, broad spectrum products, and some isolated cannabinoid products, have very limited consumer appeal to those who do not like or cannot tolerate the unique organoleptic properties. As a consequence, there is a large segment of the population who cannot take advantage of therapeutic benefits of cannabinoid containing compositions due to the negatively perceived organoleptic properties. Furthermore, there is a particular challenge to develop efficacious cannabinoid compositions that have wide appeal for therapeutic use. While these products have great potential to treat a broad range of health issues, they suffer from undesirable taste and smell due to the nature of natural terpenes and other chemistries associated with cannabis. As a result, numerous formulations exist that try to mask or hide the distinctive organoleptic character of cannabis. The draw back with these formulations whether ingested or applied topically is that they do not completely mitigate the negative organoleptic properties and as a result limit product appeal and overall usability. Furthermore, the use of additional chemistries to alter or overcome perceived negative aspects of broad spectrum and non-broad spectrum cannabis compositions is undesirable. This is namely due to the market place demand for additive free, pure natural products. Furthermore, there are limitations of additive types as per potential regulatory requirements commensurate with product use, further limiting the ability to overcome the negative aspects of natural cannabis-based products and compositions. To that end, there is great interest and commercial value in delivering compositions that contain broad spectrum cannabinoids or singular cannabinols with pleasant, appealing organoleptic properties while maintaining therapeutic effects.

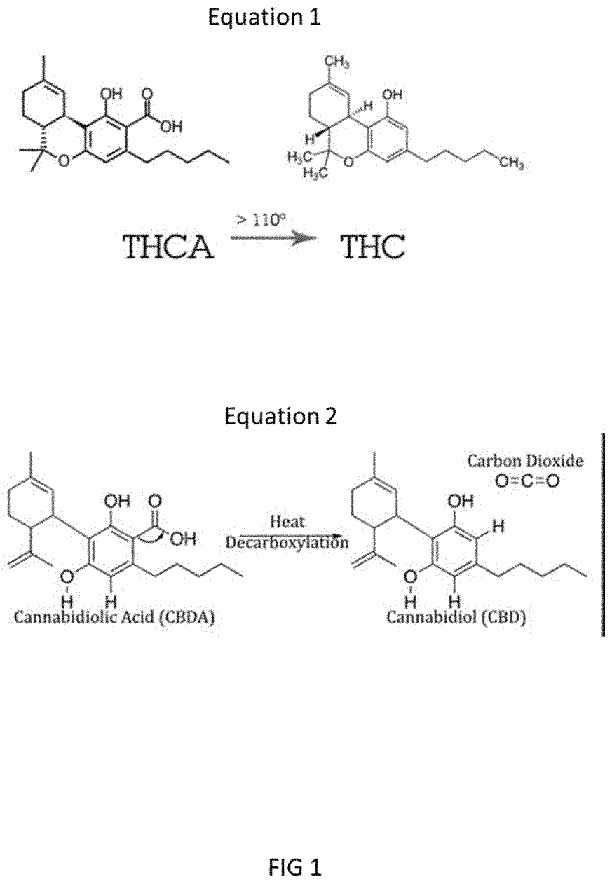

It is known that heating cannabis and or hemp can produce specific forms of decarboxylated THC and CBD following the generalized decarboxylation reactions shown in FIG. 1, Equation 1 and Equation 2. The decarboxylated forms of THC and CBD are desirable due to their psychoactive and therapeutic properties. Decarboxylation is a thermal process involving intra molecular condensation reactions that liberate carbon dioxide, i.e., cannabidiolic acid (CBDA) forms cannabidiol (CBD) and tetrahydrocannabinolic acid (THCA) form tetrahydrocannabinol (THC) from the conversion of a carboxylic acid group to form carbon dioxide. Other cannabinoids found in hemp or cannabis can undergo similar, thermally induced decarboxylation reactions which may have significant therapeutic value, as monotherapy or in combination with other therapeutic agents.

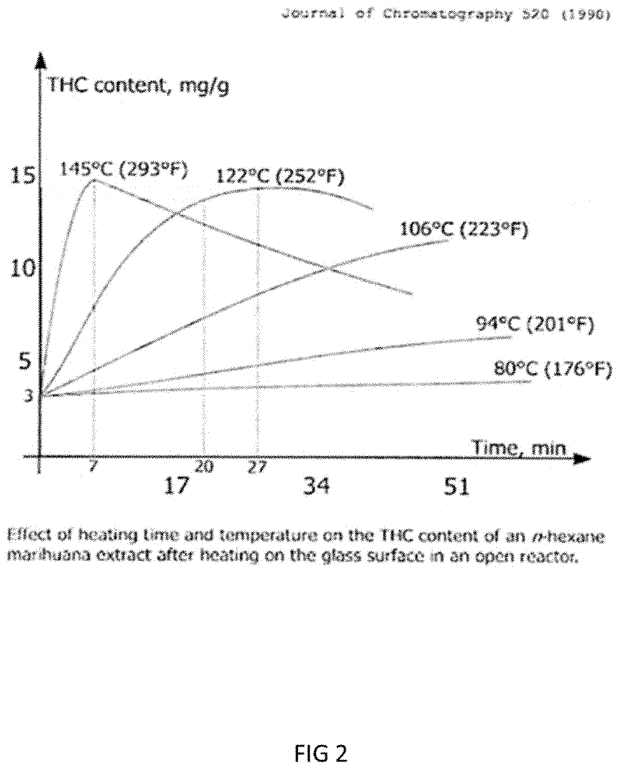

Furthermore, prior art teaches generalized temperature and exposure times at temperature to produce decarboxylated THC and CBD under normal atmospheric conditions. FIG. 2 illustrates the general effect of heating time and temperature on THC content. It is important to note that the THC content reaches a maximum, then decreases at each temperature level. This is primarily due to oxidative susceptibility of THC, CBD, and other cannabinoids

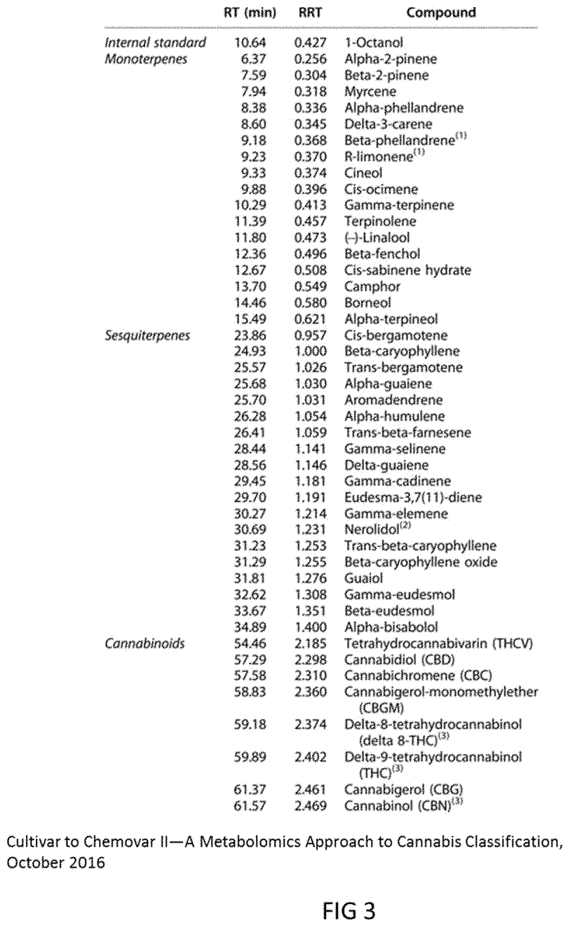

Furthermore, research continues to progress at a rapid pace to evaluate and classify different cultivars, such as Sativa and Indica types, based on their chemical content and therapeutic efficacy to treat different illnesses ranging from chronic pain and multiple sclerosis (MS) to epilepsy to anxiety. To date, there are over 700 cultivars of cannabis that have been classified. While each of these cultivars has a given level of THC, CBD and other known cannabinoids, of particular interest are the terpenoid and flavonoid profiles and contents of different cannabis cultivars. It has become increasingly clear that multiple constituents may be involved in the overall efficacy of the varietal type. To that end, over 120 different terpene types have been identified in cannabis and may have therapeutic value. While not wishing to be bound by theory, the terpene types may be involved in regulating THC and other cannabinoids. FIG. 3 illustrates a list of exemplary terpenes contained in cannabis.

Terpenoids constitute the largest class of natural products derived from isoprene (C5) units joined head-to-tail or tail-to-head, among other possibilities. They are classified as hemiterpenes (C5), monoterpenes (C10), sesquiterpenes (C15), diterpenes (C20), sesterpenes (C25), triterpenes (C30), tetraterpenes (C40), and polyterpenes (>C40). They can be found in numerous living organisms, especially plants, fungi, and marine animals. Terpenoids are of great interest due to the broad range of biological activities reported such as cancer preventive effects and analgesic, anti-inflammatory, antimicrobial, antifungal, antiviral, and antiparasitic activities. Flavonoids are hydroxylated phenolic compounds that are present in plants and occupy a special place among secondary metabolites. They are classified into different classes, with flavones, flavonols, flavanones, catechins, isoflavones, and anthocyanidins being the most common. Similar to terpenoids, they also present a wide range of biological activities. These compounds have been demonstrated to have protective effects against many infectious and degenerative diseases such as cancer, among other important pharmacological activities such as antioxidant and anti-inflammatory activities (Potential of Terpenoids and Flavonoids from Asteraceae as Anti-Inflammatory, Antitumor, and Antiparasitic Agents, Evid. Based Complement Alternat. Med. 2017; 2017: 6196198). To that end there is great interest in extracting desirable levels and combination of flavonoids and terpenoids in conjunction with THC and CBD. It is understood that terpenoids and flavonoids are typically volatile and as such can easily vaporize and are difficult to capture during extraction processes known in the art. Furthermore, elaborate processes that potentially retain some level of terpenoids and flavonoids typically produce extracts and compositions that suffer from undesirable smell and taste and as such are not readily accepted.

In addition, the protein and natural sugar content of cannabis is of interest and relevant to the disclosure described herein. Since the discovery of the endocannabinoid system in the human body and recent evidence of cannabis' therapeutic benefits to treat illnesses, protein analysis has become a current topic of research. (Optimization of Protein Extraction from Medicinal Cannabis Mature Buds for Bottom-Up Proteomics Molecules 2019, 24(4), 659) Wherein, protein extracts from apical buds and trichomes overall generated 26,892 intact protein LC-MS peaks (ions), which were then clustered into 5,408 isotopic clusters, which were in turn grouped into 571 proteins of up to 11 charge states. To that end, the natural biochemistry of cannabis in general, has been found to be useful in creating new organoleptic characteristics when processed according to the disclosure described herein.

It has been unexpectedly discovered that rapid heating of cannabis material to temperatures of 150° C. to 350° C. under an inert atmosphere produces decarboxylated forms of THC and CBD in much shorter times and greater efficiency that disclosed in prior art. In addition, heating in an inert atmosphere at a sufficient flow rate in a closed chamber entrains volatile terpenoids and flavonoids which can be extracted from the vapor using an appropriate medium, which can further be used in compositions, mixtures, and products suitable for ingestion, inhalation, or topical application. Furthermore, it has been discovered that heating cannabis, or mixtures of cannabis plant material, in an inert atmosphere, can be used to produce desirable organoleptic characteristics when extracted using extraction techniques, but not limited to, ethanol, butane, propane, other high-vapor-pressure solvents, super critical CO₂ or combinations thereof, which can in turn be used to in compositions, mixtures, and products suitable for ingestion, inhalation, or topical application. Furthermore, it has been discovered that extracts from thermally treated cannabis plant material and extracts derived from capturing volatile terpenoid and flavonoid during thermal treatment (i.e., heating) of cannabis plant material can be combined to create a mixture suitable for use in compositions, mixtures, and products suitable for use in inhalation, ingestion, or topical applications. In addition, it has been discovered that extraction medium can contain mixtures of organic solvents and medium specifically useful for inhalation, ingestion, or topical application wherein, the organic solvents can be removed to further concentrate extracted elements from the cannabis plant material.

It has also been discovered that cannabis plant material treated with organic acids or bases prior to heat treatment produces exceptional organoleptic characteristics and further solubilizes otherwise insoluble components in organic solvents (or medium) with a varying degree of polarity. Details and examples according to the disclosure disclosed herein are described in the following section pertaining to process, equipment design, chemical composition, and application of the unique chemical compositions produced using the process, methods, and designs disclosed herein.

A novel process has been discovered and developed to produce unique compositions that contain specific mass ratios of THC, CBD, terpenoids, flavonoids, thermally altered flavonoids and terpenes extracted from cannabis plant material and mixtures of cannabis plant materials of differing varietal type. The disclosure described herein discloses cannabinoid containing compositions with exceptional organoleptic properties and therapeutic properties that are superior to and overcome the well-known drawbacks of known cannabis extracts, compositions, and mixtures used for inhalation, ingestion, and topical applications. The chemical compositions created as result of the process disclosed herein, are suitable and have application to various modes of delivery and treatment to increase their usability, appeal, and therapeutic value to treat specific illnesses. Specifically, the chemical nature of the compositions are described according to the disclosure are disclosed. Furthermore, scale-able process methods, equipment design, and steps necessary to enable the disclosure for industrial production and for point of sale processing to create extracts and compositions containing cannabinoids containing compositions in accordance with the disclosure are described. Furthermore, extraction compositions, made in accordance with the disclosure, have further applications in consumer products, remedies, pharmaceutical products or other industrial applications, such as additive to improve the overall organoleptic perception of inferior plant stocks, reduce the variability of organoleptic properties, and impart desirable organoleptic properties or therapeutic benefits.

Provided herein are, for example, compositions comprising cannabinoid containing materials that have been thermally treated to have certain organoleptic properties. Also provided herein are, for example, methods of delivering thermally treated cannabinoid containing materials.

The disclosure and the various features and advantageous details thereof are explained more fully with reference to the non-limiting embodiments and examples that are described and/or illustrated in the accompanying drawings and detailed in the following. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure.

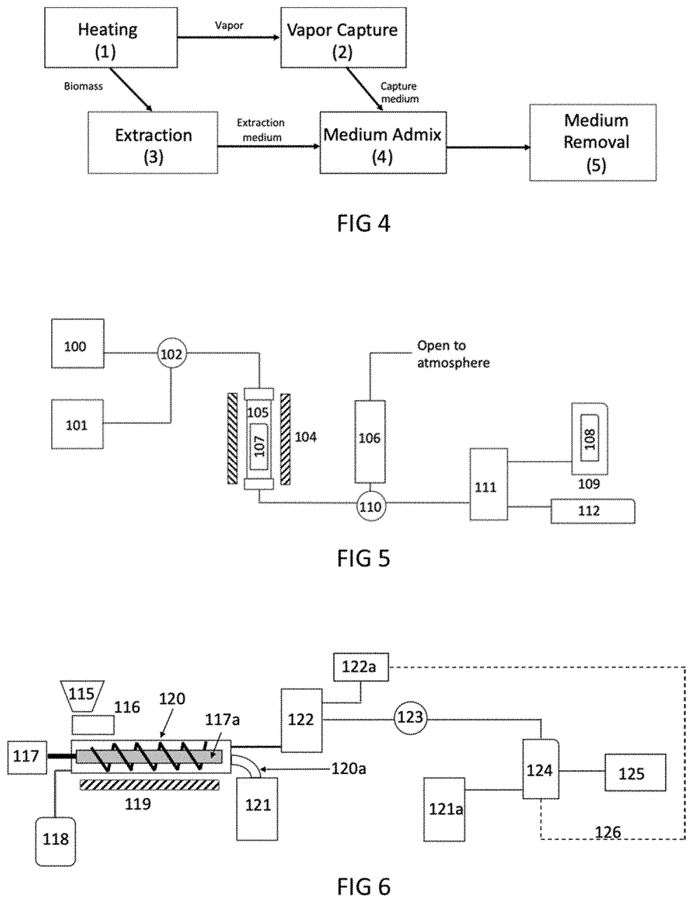

The following describes methods and processes to create the unique chemical compositions containing desirable cannabinoids, terpenoids, and flavonoids described herein. The utility of the process has essentially, but is not limited to, five primary blocks: 1) heating plant material in a sub-stoichiometric oxygen atmosphere wherein the atmosphere is flowing in a controlled manner, 2) removal of volatile components entrained in the flowing atmosphere using at least one medium and at least one temperature condition, 3) extraction of heated plant material described in block 1, 4) combining the liquid described in block 2 with the extract described in block 3, 5) removal of processing liquids from the composition described in block 4. It is understood that any of the described process elements described can be used singularly, in combination, or simultaneously to produce compositions in accordance with the disclosure described herein. It is understood that the term liquid is not bound by standard, temperature, and pressure conditions, but can be any medium suitable to capture, dissolve, suspend or otherwise form a mixture or composition wherein the medium can be separated from the desired components. It is also understood that the term medium denotes liquids, gases, solids or combinations thereof. The term extraction is a non-limiting term to describe non limiting extraction techniques such as alcohol, CO₂, butane, propane, steam, water, other organic solvents used singularly or in combination. The generalized process flow is illustrated in FIG. 4.

Furthermore, suitable plant-based feedstock can consist of essentially any part of, but not limited to, the cannabis plant such as flower, leaves, stems, stalks and any part of the plant use singularly or in combination with other parts of the plant or combinations with other varietal types. In addition, combinations of cannabis plant and non-cannabis plant type can be selected to achieve specific organoleptic characteristics. The plant material can be physically altered or modified to enhance the efficiency of the process, for example but not limited to, granulated, pulverized, chopped, reconstituted, or further processed such as expanded using, but not limited to, CO2, densified, or any combination thereof. Furthermore, the term plant material can denote compositions of plant material such as, but not limited to, cannabis and other chemistries in the form of liquids, solids, or gas(es) or any combination thereof, to promote desirable thermal reactions with plant material during operation of the process. The term sub-stochiometric atmosphere denotes, but is not limited to, an atmosphere essentially devoid of oxygen or containing a level of oxygen below normal atmospheric compositions containing oxygen. In addition, the atmosphere can be comprised of a singular composition or a plurality of gaseous components, including water vapor or other organic vapors, used in a static state or a flowing state, where the flowing state, denoted as a flow rate, can be of a singular or varied value during the course of using the process. It is also understood that the process can be operated at, below, or above standard atmospheric conditions. For example, the process addresses the problem that users who regularly smoke cannabis report; consuming non-thermally treated extracts does not provide the same effect, taste and satisfaction as smoking does. However, due to the sub-stochiometric process conditions, the plant material is not subjected to oxidative stress and thus, undesirable byproduct molecules typically produced by combustion are not created.

Additional Embodiments include Embodiments 1 to 145 following.

Embodiment 1. A cannabis composition, comprising at least one cannabinoid and at least one flavonoid.

Embodiment 2. The composition of embodiment 1, wherein the at least one flavonoid is a bioflavonoid, an isoflavonoid, a neoflavonoid, or combinations thereof.

Embodiment 3. The composition of embodiment 1 or 2, wherein the at least one flavonoid is a natural flavonoid, a synthetic flavonoid, or a combination thereof.

Embodiment 4. The composition of any one of embodiments 1-3, wherein the at least one flavonoid is a natural flavonoid.

Embodiment 5. The composition of any one of embodiments 1-4, wherein the at least one cannabinoid is a natural cannabinoid, a synthetic cannabinoid, or a combination thereof.

Embodiment 6. The composition of embodiment 5, wherein the at least one cannabinoid is a natural cannabinoid.

Embodiment 7. The composition of any one of embodiments 1-6, wherein the composition further comprises at least one terpenoid and/or terpene.

Embodiment 8. The composition of claim 7, wherein the at least one terpenoid and/or terpene is a natural terpenoid and/or terpene, a synthetic terpenoid and/or terpene, or a combination thereof.

Embodiment 9. The composition of embodiment 8, wherein the at least one terpenoid and/or terpene is a natural terpenoid and/or terpene.

Embodiment 10. The composition of embodiment 8, wherein the composition is derived or extracted from a plant material.

Embodiment 11. The composition of embodiment 11, wherein the composition is fully derived or extracted or partially derived or extracted from a plant substrate.

Embodiment 14. The composition of embodiment 10 or 11, wherein the plant substrate is plant material.

Embodiment 13. The composition of embodiment 12, wherein the plant material is plant flowers, plant leaves, plant stems, plant stalks, or combinations thereof.

Embodiment 14. The composition of embodiment 12 or 13, wherein the plant material is a cannabis plant, a hemp plant, or a combination thereof.

Embodiment 15. The composition of embodiment 14, wherein the cannabis plant material comprises at least one species from the cannabaceae family.

Embodiment 16. The composition of any one of embodiments 11-15, wherein the composition is derived by exposing the plant substrate to heat under a gas atmosphere.

Embodiment 17. The composition of embodiment 17, wherein the plant substrate is heated to a temperature of from about 100° C. to about 500° C.

Embodiment 18. The composition of embodiment 25 or 26, wherein the plant substrate is heated to a temperature of from about 150° C. to about 350° C.

Embodiment 19. The composition of any one of embodiments 16-18, wherein the gas environment comprises at least one of nitrogen gas, argon gas, carbon dioxide gas, helium gas, or oxygen gas.

Embodiment 20. The composition of any one of embodiments 16-19, wherein the gas environment comprises up to about 21% oxygen by weight.

Embodiment 21. The composition of any one of embodiments 16-20, wherein the gas environment comprises up to about 15% oxygen by weight.

Embodiment 22. The composition of any one of embodiments 16-21, wherein the gas environment comprises up to about 10% oxygen by weight.

Embodiment 23. The composition of any one of embodiments 16-22, wherein the gas environment is essentially free of oxygen.

Embodiment 24. The composition of any one of embodiments 16-22, further comprising a reaction vessel.

Embodiment 25. The composition of embodiment, 24, wherein the reaction vessel is a closed system vessel.

Embodiment 26. The composition of any one of embodiments 16-25, wherein the gas environment is at atmospheric pressure, below atmospheric pressure, or above atmospheric pressure.

Embodiment 27. The composition of any one of embodiments 16-26, wherein the gas environment comprises a gas flow.

Embodiment 28. The composition of any one of embodiments 16-27, wherein the gas environment comprises a continuous gas flow.

Embodiment 29. The composition of embodiment 28, wherein the non-stationary gas flow is a gas stream.

Embodiment 30. The composition of any one of embodiments 1-29, wherein the at least one cannabinoid comprises a cannabigerol (CBG), a cannabichromene (CBC), a cannabidiol (CBD), tetrahydrocannabinol (THC), cannabinol (CBN), cannabielsoin (CBE), iso-tetrahydrocannabinol (iso-THC), cannabicyclol (CBL), cannabicitran (CBT), CBG-type cannabinoid a CBC-type cannabinoid, a CBD-type cannabinoid, a THC-type cannabinoid, a CBN-type cannabinoid, a CBE-type cannabinoid, an iso-THC-type cannabinoid, a CBL-type cannabinoid, a CBT-type cannabinoid, a CBG-type cannabinoid, or combinations thereof.

Embodiment 31. The composition of any one of embodiments 1-30, wherein the at least one cannabinoid comprises THC, CBD, CBC, CBN, CBG, CBL, a THC-type cannabinoid, a CBD-type cannabinoid, a CBC-type cannabinoid, a CBN-type cannabinoid, a CBG-type cannabinoid, a CBL-type cannabinoid, or combinations thereof.

Embodiment 32. The composition of embodiment 30 or 31, wherein the THC-type cannabinoid is tetrahydrocannabinolic acid (THCA), tetrahydrocannabivarin (THCV), Δ⁹-THC, Δ⁸-THC, 8-hydroxy-Δ⁹-tetrahydrocannabinol, or combinations thereof.

Embodiment 33. The composition of embodiment 32, wherein the THC-type cannabinoid is Δ⁸-THC.

Embodiment 34. The composition of any one of embodiments 31-33, wherein the at least one cannabinoid comprises a CBC-type cannabinoid, cannabidiolic acid (CBGA), cannabichromene, a CBC-type cannabinoid, CBD, or combinations thereof.

Embodiment 35. The composition of embodiment 34, wherein the cannabinoid is CBD.

Embodiment 36. The composition of any one of embodiments 1-35, wherein the composition further comprises a fatty acid or ester thereof.

Embodiment 37. The composition of embodiment 36, wherein the fatty acid or ester thereof comprises a short- or medium-chain fatty acid or ester thereof.

Embodiment 38. The composition of embodiment 36 or 37, wherein the fatty acid ester is a C₁-C₆ ester.

Embodiment 39. The composition of any one of embodiments 36-38 wherein the fatty acid ester is a methyl ester.

Embodiment 40. The composition of any one of embodiments 1-39, wherein the composition further comprises: 2,2-dimethyloxetane; pentanal; dimethyl disulfide; pyridine; 1-acetylcyclohexene; acetamide; 3-furaldehyde; p-mentha-1,3,8-triene; p-mentha-1,5,8-triene; 6-methyl-5-hepten-2-one; 2,4-dimethyl-2,6-heptadien-1-ol; benzaldehyde; butyrolactone; (1R,5S)-2-methylene-6,6-dimethylbicyclo[3.1.1]heptane; p-(1-propenyl)-toluene; p-cymenene; acetophenone; caryophyllene; humulene; 2-isopropenyl-4a,8-dimethyl-1,2,3,4,4a,5,6,7-octahydronaphthalene; alpha-guaiene; 1,2-benzenediol, [2-(2-methylpropoxycarbonyloxy)phenyl] 4-butylbenzoate; (8aR)-8a-methyl-4-methylidene-6-propan-2-ylidene-2,3,4a,5,7,8-hexahydro-1H-naphthalene; neophytadiene; 1,4-dimethyl-7-prop-1-en-2-yl-1,2,3,3a,4,5,6,7-octahydroazulene; cannabichromene; (6aR,8R,10aR)-6,6,9-trimethyl-3-pentyl-6a,7,8,10a-tetrahydrobenzo[c]chromene-1,8-diol; 1H-tetrazole; allyl acetate; (1R)-2,6,6-trimethylbicyclo[3.1.1]hept-2-ene; (1R,5S)-2-methylene-6,6-dimethylbicyclo[3.1.1]heptane; 2,3-dihydroxypropyl acetate; (1aS,3aR,8bR,8R)-1,1,3a-trimethyl-6-pentyl-1a,2,3,3a,8b,8c-hexahydro-1H-4-oxabenzo[f]cyclobuta[cd]inden-8-ol; dronabinol; 7-isopropyl-4a,8a-dimethyloctahydro-1(2H)-naphthalenone; n-propyl 9,12-octadecadienoate; methyl 9,12,15-octadecatrienoate; 6,9 octadecadienoic acid methyl ester; ethyl linolenate; butyl 9,12-octadecadienoate; 9,12-octadecadienoyl chloride (z,z); methyl 9-cis, 11 trans-octadecadienoate; 2-mono-linolein; methyl (11E,14E)-octadeca-11,14-dienoate; ethyl linolenate; methyl heptadeca-8,11,14-trienoate; methyl (9Z,12Z,15Z)-2-hydroxyoctadeca-9,12,15-trienoate; methyl (7E,10E,13E)-hexadeca-7,10,13-trienoate; butyl (9Z,12Z,15Z)-9,12,15-octadecatrienoate; methyl (5Z,8Z,11Z,14Z)-icosa-5,8,11,14-tetraenoate; n-propyl 9,12,15-octadecatrienoate, or combinations thereof.

Embodiment 41. The composition of any one of embodiments 1-40, wherein the composition further comprises at least one carrier, excipient, or additive.

Embodiment 42. The composition of embodiment 41, wherein the at least one carrier, excipient, or additive is selected from diluents, antiadherents, binders, coatings, disintegrants, surfactants, dissolving agents, solubilizing agents, bioadhesive agents, polysaccharides, polymers, copolymers, bioavailability enhancing agents, thin film-type excipient, mucoadhesive agents, acidifying agents, probiotic agents, protective agents, antioxidants, dispersing agents, flavors, color additives, fragrance, lubricants, glidants, sorbents, preservatives, sweeteners, or combinations thereof.

Embodiment 43. The composition embodiment 41 or 42, wherein the at least one carrier, excipient, or additive is at least one solvent or solvent system.

Embodiment 44. The composition of embodiment 43, wherein the at least one solvent or solvent system comprises an organic solvent, an inorganic solvent, or a combination thereof.

Embodiment 45. The composition of embodiment 43 or 44, wherein the at least one solvent or solvent system comprises a polar solvent, a non-polar solvent, or a combination thereof.

Embodiment 46. The composition of any one of embodiments 43-45, wherein the composition is miscible with the solvent or solvent system.

Embodiment 47. The composition of any one of embodiments 43-46, wherein the composition is immiscible with the solvent or solvent system.

Embodiment 48. The composition of any one of embodiments 43-47, wherein the at least one solvent or solvent system comprises an oil.

Embodiment 49. The composition of embodiment 48, wherein the oil is MCT oil, olive oil, canola oil, hemp oil, or combinations thereof.

Embodiment 50. The composition of any one of embodiments 43-49, wherein the at least one solvent is or solvent system comprises water, alcohol, a hydrocarbon, an ether, or combinations thereof.

Embodiment 51. The composition of embodiment 50, wherein the alcohol is ethanol, propylene glycol, glycerol, or combinations thereof.

Embodiment 52. The composition of embodiment 50 or 51, wherein the hydrocarbon is a fluorinated hydrocarbon.

Embodiment 53. The composition of embodiment 52, wherein the fluorinated hydrocarbon is tetrafluoroethylene, propane, butane, or a combination thereof.

Embodiment 54. The composition of any one of embodiments 50-54, wherein the ether is polyethylene glycol (PEG).

Embodiment 55. The composition of embodiment 54, wherein the ether is polyethylene glycol 400 (PEG 400).

Embodiment 56. The composition of any one of embodiments 43-55, further comprising a co-solvent.

Embodiment 57. The composition of any one of embodiments 1-62, wherein the composition further comprises at least one preservative.

Embodiment 58. The composition of any one of embodiments 1-57, wherein the composition is in the form of an oil, a liquid, a solid, or a gas.

Embodiment 59. The composition of embodiment 58, wherein the liquid is a solution, a suspension, or an emulsion.

Embodiment 60. The composition of any one of embodiments 1-59, wherein the composition is in the form of a paste, a cream, a gel, a liniment or balm, an aerosol, a lotion, an ointment, drops, a concentrate, a skin patch, an oral or nasal spray, a film, a food or beverage additive, an edible food product, a tablet, a capsule, a fast dissolving tablet (FDT), an effervescent tablet, a syrup, an elixir, a cartridge, or a suppository.

Embodiment 61. The composition of any one of embodiments 1-60, wherein the composition is formulated for oral delivery, topical delivery, enteral delivery, parenteral delivery, intranasal delivery, sublingual delivery, buccal delivery, inhalation delivery.

Embodiment 62. The composition of embodiment 61, wherein the inhalation delivery is inhalation by mouth, inhalation by nose, or a combination thereof.

Embodiment 63. The composition of embodiment 61 or 62, wherein the inhalation delivery is by inhaler, nebulizer, vaporizer, aerosolizer, or a smoking device.

Embodiment 64. The composition of embodiment 63, wherein the smoking device is a cigarette, cigar, pipe, or an electronic smoking device.

Embodiment 65. The composition of embodiment 64, wherein the smoking device is a cigarette or cigar comprising tobacco, hemp cannabis, herbs, spices, or combinations thereof.

Embodiment 66. The composition of embodiment 64, wherein the device is an electronic smoking device.

Embodiment 67. The composition of embodiment 64, wherein the vaporizer is an open system, a semi-open system, or closed system vaporizer.

Embodiment 68. The composition of any one of embodiments 1-67, wherein the composition has a peak with a retention time of about 6.6 min in a GC-MS chromatogram.

Embodiment 69. The composition of any one of embodiments 1-68, wherein the composition has at least one additional peak with a retention time of about 4.5, about 4.6, about 4.7, about 4.8, about 4.9, or about 7.15 minutes in a GC-MS chromatogram.

Embodiment 70. The composition of any one of embodiments 1-69, wherein the composition has at least two additional peaks with a retention time of about 4.5, about 4.6, about 4.7, about 4.8, about 4.9, or about 7.15 minutes in a GC-MS chromatogram.

Embodiment 71. The composition of any one of embodiments 1-70, wherein the composition has at least three additional peaks with a retention time of about 4.5, about 4.6, about 4.7, about 4.8, about 4.9, or about 7.15 minutes in a GC-MS chromatogram.

Embodiment 72. The composition of any one of embodiments 1-71, wherein the composition has at least four additional peaks with a retention time of about 4.5, about 4.6, about 4.7, about 4.8, about 4.9, or about 7.15 minutes in a GC-MS chromatogram.

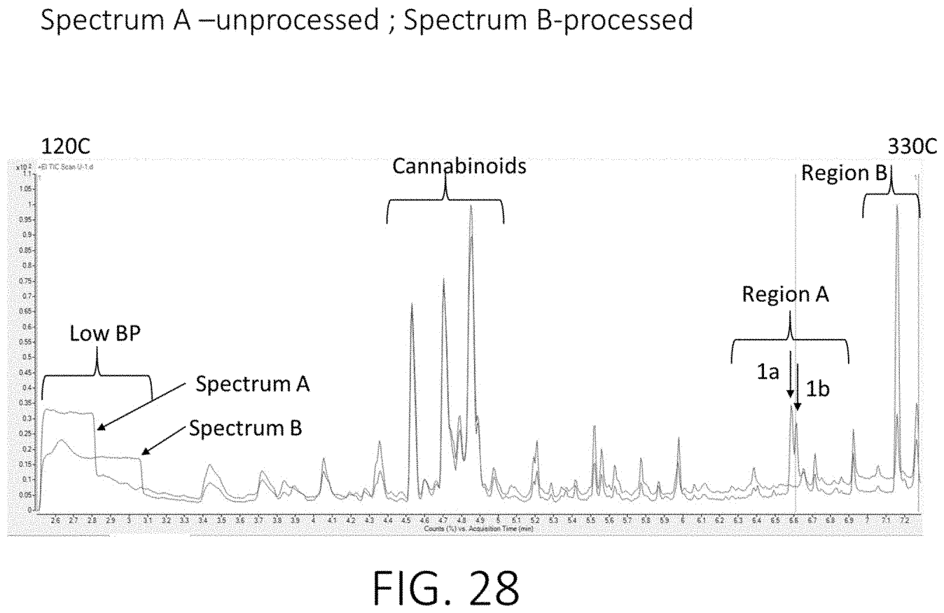

Embodiment 73. The composition of any one of embodiments 1-72, wherein the composition is characterized by a GC-MS chromatogram of FIG. 28.

Embodiment 74. A process for producing the composition of any one of embodiments 1-73, comprising:
(i) providing an unprocessed plant feedstock in a gas environment;
(ii) heating the unprocessed plant feedstock in the gas environment to afford a processed plant product and at least one volatile compound; and
(iii) extracting the treated plant product of (ii) with a first solvent or solvent system thereby providing a first extract

Embodiment 75. The process of embodiment 74, wherein the plant material is heated to a temperature of from about 100° C. to about 500° C.

Embodiment 76. The process of embodiment 74 or 75, wherein the plant material is heated to a temperature of from about 150° C. to about 350° C.

Embodiment 77. The process of any one of embodiments 74-76, wherein the gas environment comprises at least one of nitrogen gas, argon gas, carbon dioxide gas, helium gas, or oxygen gas.

Embodiment 78. The process of any one of embodiments 74-77, wherein the gas environment comprises up to about 21% oxygen by weight.

Embodiment 79. The process of any one of embodiments 74-78, wherein the gas environment comprises up to about 15% oxygen by weight.

Embodiment 80. The process of any one of embodiments 74-79, wherein the gas environment comprises up to about 10% oxygen by weight.

Embodiment 81. The process of any one of embodiments 74-80, wherein the gas environment is essentially free of oxygen.

Embodiment 82. The process of any one of embodiments 74-81, wherein the gas environment is at atmospheric pressure, below atmospheric pressure, or above atmospheric pressure.

Embodiment 83. The process of any one of embodiments 74-82, wherein the gas environment comprises a gas flow.

Embodiment 84. The process of embodiment 83, wherein the gas flow is a gas stream.

Embodiment 85. The process of embodiment 84, wherein the gas flow is pressurized gas.

Embodiment 86. The process of any one of embodiments 74-85, wherein the first solvent or solvent system comprises an organic solvent, an inorganic solvent, or a combination thereof.

Embodiment 87. The process of any one of embodiments 74-86, wherein the first solvent or solvent system comprises a polar solvent, a non-polar solvent, or a combination thereof.

Embodiment 88. The process of any one of embodiments 74-87, wherein the first solvent or solvent system comprises water, alcohol, a hydrocarbon, an ether, CO2, or combinations thereof.

Embodiment 89. The process of embodiment 88, wherein the alcohol is ethanol.

Embodiment 90. The composition of 88 or 89, wherein the hydrocarbon is a fluorinated hydrocarbon.

Embodiment 91. The process of embodiment 98, wherein the fluorinated hydrocarbon is tetrafluoroethylene.

Embodiment 92. The process of any one of embodiments 88-91, wherein the hydrocarbon is propane, butane, or a combination thereof.

Embodiment 93. The process of embodiment 88, wherein the CO2 is supercritical CO2, sub-critical CO2, or a combination thereof.

Embodiment 94. The process of any one of embodiments 74-93, further comprising a first co-solvent.

Embodiment 95. The process of any one of embodiments 74-94, wherein the first solvent or solvent system is mono-phasic or bi-phasic.

Embodiment 96. The process of any one of embodiments 74-95, wherein the first extract is concentrated.

Embodiment 97. The process of embodiment 96, wherein the first extract is concentrated by evaporation at atmospheric pressure, evaporation under vacuum, or a combination thereof.

Embodiment 98. The process of embodiment 96 or 97, wherein the first extract is concentrated by application of heat.

Embodiment 99. The process of any one of embodiments 74-98, wherein the process further comprises combining the first extract with a first additive.

Embodiment 100. The process of embodiment 99, wherein the first additive is ethanol, propylene glycol, glycerol, PEG400, MCT oil, olive oil, canola oil, hemp oil, cannabis oil, or combinations thereof.

Embodiment 101. The process of any one of embodiments 74-100, wherein the process further comprises capturing and extracting the at least one volatile compound with a second solvent or solvent system, an adsorbent substrate, or a combination thereof thereby providing a second extract.

Embodiment 102. The process of embodiment 101, wherein the second solvent or solvent system comprises an organic solvent, an inorganic solvent, or a combination thereof.

Embodiment 103. The process of embodiment 101 or 102, wherein the second solvent or solvent system comprises a polar solvent, a non-polar solvent, or a combination thereof.

Embodiment 104. The process of any one of embodiments 101-103, wherein the second solvent or solvent system comprises water, alcohol, a hydrocarbon, an ether, CO2, or combinations thereof.

Embodiment 105. The process of any one of embodiments 101-104, wherein the second solvent or solvent system comprises an oil.

Embodiment 106. The process of embodiment 105, wherein the oil comprises an MCT oil, olive oil, canola oil, hemp oil, or combinations thereof.

Embodiment 107. The process of any one of embodiments 104-106, wherein the alcohol is ethanol, propylene glycol, glycerol, or combinations thereof.

Embodiment 108. The process of any one of embodiments 104-107, wherein the hydrocarbon is a fluorinated hydrocarbon.

Embodiment 109. The process of embodiment 108, wherein the fluorinated hydrocarbon is tetrafluoroethylene.

Embodiment 110. The process of any one of embodiments 104-109, wherein the hydrocarbon is propane, butane, or a combination thereof.

Embodiment 111. The process of any one of embodiments 104-110, wherein the ether is polyethylene glycol (PEG).

Embodiment 112. The process of any one of embodiments 104-111, wherein the ether is polyethylene glycol 400 (PEG 400).

Embodiment 113. The process of embodiment 104, wherein the CO2 is supercritical CO2, sub-critical CO2, or a combination thereof.

Embodiment 114. The process of any one of embodiments 101-113, further comprising a second co-solvent.

Embodiment 115. The process of any one of embodiments 101-114, wherein the second solvent or solvent system is mono-phasic or bi-phasic.

Embodiment 116. The process of any one of embodiments 101-115, wherein the second extract is concentrated.

Embodiment 117. The process of embodiment 116, wherein the second extract is concentrated by evaporation at atmospheric pressure, evaporation under vacuum, or a combination thereof.

Embodiment 118. The process of embodiment 116 or 117, wherein the second extract is concentrated by application of heat.

Embodiment 119. The process of any one of embodiments 74-118, wherein the process comprises entraining the at least one volatile compound in a flowing gas stream.

Embodiment 120. The process of embodiment 119, wherein the flowing gas stream comprises at least one of nitrogen gas, argon gas, carbon dioxide gas, helium gas, or oxygen gas.

Embodiment 121. The process embodiment 119 or 120, wherein the flowing gas stream comprises up to about 21% oxygen by weight.

Embodiment 122. The process any one of embodiments 119-121, wherein the flowing gas stream comprises up to about 15% oxygen by weight.

Embodiment 123. The process any one of embodiments 119-122, wherein the flowing gas stream comprises up to about 10% oxygen by weight.

Embodiment 124. The process any one of embodiments 119-123, wherein the flowing gas stream is essentially free of oxygen by weight.

Embodiment 125. The process of any one of embodiments 119-124, wherein the process further comprises combining the second extract with a second additive.

Embodiment 126. The process of embodiment 125, wherein the second additive is ethanol, propylene glycol, glycerol, polyethylene glycol 400 (PEG400), MCT oil, olive oil, canola oil, hemp oil, cannabis oil, or combinations thereof.

Embodiment 127. The process of any one of embodiments 101-126, wherein the first extract is combined with the second extract.

Embodiment 128. The process of any one of embodiments 74-127, further comprising combining the unprocessed plant feedstock of (i) with an additive.

Embodiment 129. The process of embodiment 128, wherein the additive is a pH adjusting agent, a protein, a carbohydrate, or combinations thereof.

Embodiment 130. The process of embodiment 129, wherein the pH adjusting agent is a bicarbonate, an alkali metal hydroxide, an amine, ammonia, or combinations thereof.

Embodiment 131. The process of embodiment 130, wherein the bicarbonate is sodium bicarbonate.

Embodiment 132. The process of embodiment 131, wherein about 0.5% to about 25% by weight of sodium bicarbonate is added to the unprocessed plant feedstock of (i).

Embodiment 133. The process of embodiment 132 or 132, wherein about 2% to about 15% by weight of sodium bicarbonate is added to the unprocessed plant feedstock of (i).

Embodiment 134. The process of any one of embodiments 74-133, wherein a pH adjusting agent is added to the gas environment and/or the flowing gas stream.

Embodiment 135. The process of embodiment 134, wherein the pH adjusting agent is a bicarbonate, an alkali metal hydroxide, an amine, ammonia, or combinations thereof.

Embodiment 136. The process of embodiment 135, wherein the bicarbonate is sodium bicarbonate.

Embodiment 137. A method of treating a disease or condition, comprising administering to a subject in need thereof a therapeutically effective amount of the composition of any one of embodiments 1-73.

Embodiment 138. The method of embodiment 137, wherein the disease or condition is pain, insomnia, depression, Crohn's disease, multiple sclerosis, colitis, anxiety, post-traumatic stress disorder (PTSD), seizures, glaucoma, cancer, side effects from cancer treatment, bulimia, anorexia, obesity, a skin disorder, or nausea.

Embodiment 139. A cigarette comprising the composition of any one of embodiments 1-73.

Embodiment 140. The cigarette of embodiment 139, wherein the cigarette comprises tobacco, hemp cannabis, herbs, spices, or combinations thereof.

Embodiment 141. The cigarette of embodiment 139, wherein the cigarette is an electronic cigarette.

Embodiment 142. A vaporizer cartridge comprising the composition of any one of embodiments 1-73.

Embodiment 143. A kit comprising the composition of any one of embodiments 1-7.

Embodiment 144. The kit of embodiment 143, further comprising instructions for use.

Embodiment 145. The kit of embodiment 143 or 144, further comprising a delivery device.

### II. Examples

The following descriptions provide non-limiting embodiments of the process described herein, illustrated in FIG. 5, necessary to produce the unique compositions containing describable organoleptic and therapeutic characteristics which can further be characterized by, but not limited to, their chemical composition, user preference, mitigation of anxiety, stress, and other beneficial attributes. At least one inert gas is supplied in element 100, an optional addition gas or liquid is supplied in element 101 and connected by a control valve 102 where in the flow rates and composition can be control independently. It is understood that the term connections denote suitable tubing or other means to convey liquid, solids, gas or combinations thereof. Furthermore, control valve 102 is connected to a chamber 105, suitable for holding plant material 107. The sealed chamber is surrounded by heating elements 104 that can be controlled and are arranged to achieve temperatures and heating rates. The plant material 107 is arranged and is mechanically processed to be of an appropriate particle size to allow for sufficient gas flow around and through the plant material. The flow from element 100, and optionally element 101, pass through the chamber 105 and enter control vale 110 where in flow passes into chamber 106, and exits to atmosphere. Chamber 106 contains an appropriate solvent such as but not limited to ethanol, and optionally composition components or mixtures such as, but not limited to, vegetable-based glycerin, propylene glycol, cannabis oil, mineral oil, dipropylene glycol, or mixtures thereof.

Container 106 is configured to allow the flow of gas to pass through and comingle with the liquid such that entrained high vapor pressure terpenoids and flavonoids are essentially dissolved in the liquid. Once the desired thermal treatment, typically ranging between 150° C. to 350° C., is complete and the temperature of the plant material has cooled to below oxidation temperature, typically 50° C., the post thermally treated material 108, is placed in chamber 109. Chamber 109 contains extraction medium suitable for extracting thermally treated plant material, such as but not limited to ethanol, carbon dioxide, propane, butane, or other extraction medium. It is understood that temperature, pressure, and extraction duration can be controlled. It is further understood that the typical temperature range of thermal treatment (i.e., heating) may be further adjusted for non-STP conditions. Once sufficient extraction of thermally treated plant material has been achieved, the extract concentrate is conveyed to chamber 111. The liquid contents of chamber 106 are added to chamber 111 to create a mixture composition. Chamber 111 is equipped with, but is not limited to, a means to remove solvents or other components by means of distillation or other means known in the art to remove and recover process solvents. The final composition is conveyed to chamber 112 wherein the composition can be used as processed or further mixed with or added to compositions, used as an ingredient, or component of subsequent compositions. It is understood that variables such as plant material, particle size of the plant material, atmosphere type, composition of atmosphere type, flow rates, volumes, temperatures, heating rates, cooling rates, system pressures above and below atmospheric, extraction time, temperature pressure, compositional formulation can all be controlled and varied independently to achieve the utility described herein.

In yet another embodiment, a process can be envisioned wherein the process elements described in FIG. 5 can be operated continuously to produce compositions according to the disclosure. Useful process schemes, for example, but not limited to, are illustrated in FIG. 6. Elements include, a hopper 115 to receive plant material, a grinder 116 to reduce the plant material to a desirable particle size, a chamber 120 to contain the plant material wherein the chamber 120 is equipped with a motor 117 which is connected to, but is not limited to, a variable pitch screw 117a to convey the plant material through chamber 120. In addition, the chamber is equipped with at least one heating zone along the primary axis, however preferred embodiments consist of more than one heating zone that can operate independently. Furthermore, chamber 120 is connected to a source of inert gas 118. The gas can additional be composed of multiple gases, vapors or other reactive chemicals or non-reactive chemicals. Chamber 120 is connected to a port 120a that equipped with a valve to allow material to pass into a removable extraction chamber 121. Chamber 120 is also connected to unit operation 122 sufficient to remove organic components from flowing gas, such as but not limited to a Buchi gas scrubber. The gas scrubbing unit operation contains solvent such as ethanol or mixtures such as ethanol and vegetable-based glycerin, ethanol and cannabis oil, or other compositions to enable the disclosure as previously described. The unit operation 122 is connected to a solvent reservoir 122a that maintains a desired level of solvent mixture in the unit operation 122 as it is drawn off by pump 123 during process operation. Pump 123 is connected to a least one distillation unit operation 124 equipped with an auto feed pump to maintain liquid input levels. Yet another embodiment is envisioned consisting of tandem distillation units operated in sequentially.

In addition, the distillation unit operation is connected to thermally treated plant material receptacle 121a wherein extraction methods using ethanol, supercritical CO₂ or other means to extract thermally treated plant material are employed. The extraction medium is simultaneously fed into the element 124 are the same rate, different rates, or variable rates as the gas scrubbing medium from element 122. The distillation unit operation 124 is further equipped with the means to convey the final composition into chamber 125. Furthermore, the distillation unit 124 is further equipped with recovery means 126 to recover the volatile solvents and return them to the solvent reservoir 122a.

Different operation schemes can be envisioned to enable continuous processing modes. For example, the hopper 115 and the grinder 116 work in tandem by mechanical means to convey and introduce plant material into the pre-thermal region of chamber 120 wherein the raw plant material is rendered to a desired particle size. In turn, the plant material particle size is such that it can form an impervious plug to prevent inert gas flow back through the grinder and hopper and only allow gas flow axially through chamber 120 due to a favorable pressure differential. That is to say the granulated plant material is mechanically fed via screw type feeders at a particular rate with respect to the flow rate of the inert gas such that the granular plant material will only allow the inert gas flow along the primary axis of the reactor wherein the variable pitch screw conveyer facilitates variation in plant material densities within chamber 120. In yet another embodiment, a dual hopper process and be envisioned wherein the two hoppers work sequentially to feed the grinder and introduce plant material into the reactor in a continuous manner. A first hopper will fill with plant material while the second hopper, already filled with plant material, is isolated form the natural atmosphere, wherein the plant material in the second hopper is purged with an inert gas, or essentially inert gas, before being mechanically conveyed into the second grinder. Furthermore, reactor chamber 120 has, but is not limited to, two primary zones; a preheating zone, and a heated zone. Additional heating and cooling zones can be envisioned to allow for further thermal processing control, wherein the atmosphere of each zone can be independently controlled to achieve a desired level of thermal and oxidative reactions.

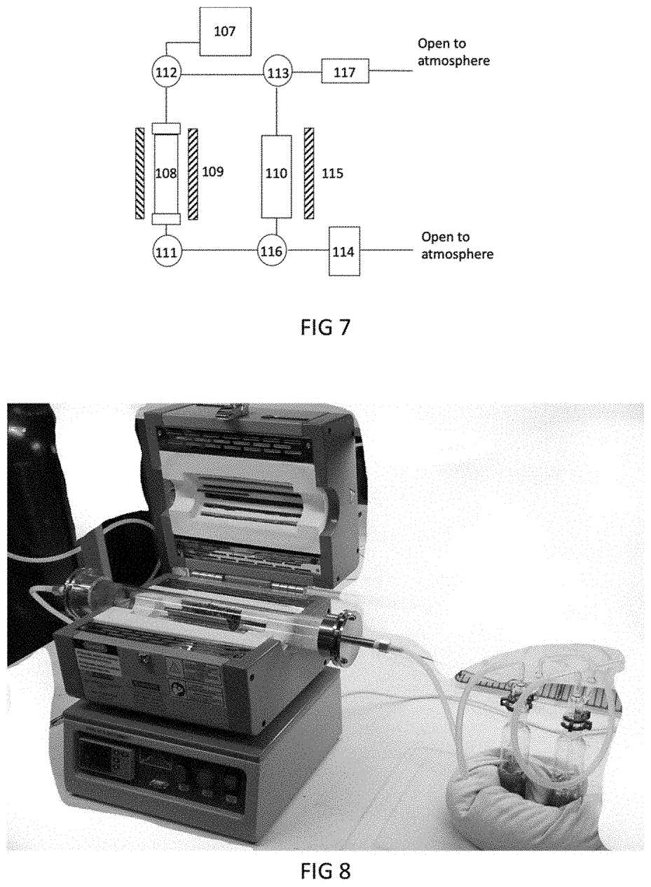

A plant material selected by consumers can be “processed” in accordance the processes and methods described herein to create compositions of the present disclosure. A proposed general two chamber processing unit is illustrated in FIG. 7. For example, a singular chamber 108 can be envisioned to receive a predetermined amount of ground plant material, ground by mechanical means either independently or a unit operation integrated into the chamber. The chamber is constructed to withstand elevated pressures and temperatures and has at least one inlet port and one outlet port. In addition, the chamber is in close proximity to thermal control elements 109 that can effectively heat the chamber and optionally cool the chamber by a number of known means. Chamber 108 is connected to valve 112 at the inlet port and valve 111 at the exit port. Valve 112 is connected to a source of inert gas such as, but not limited to, nitrogen or argon. Valve 113 is connected to a second chamber 110 equipped with at least one inlet port and at least one outlet port and a heating and cooling element 115. In addition, chamber 110 contains a medium that is suitable for extraction and gas scrubbing. In addition, chamber 110 contains a carrier such as glycerin, propylene glycol, or other suitable carriers and mixtures thereof. Furthermore, the outlet port of 110 is connected to valve 116. Finally, valve 116 is connected receptacle 114 wherein final compositions are collected. It is under stood that liquid and gas flow between the two chambers can further be optimized by integrating functions of valves 112 and 113, and valves 111 and 116.

Non-limiting examples of general operational schemes are detailed in the following descriptions. Ground plant material is placed in chamber 108 and sealed with a chamber closure. Nitrogen flow from 107 is controlled by valve 112 and as such allows the gas to pass through chamber 108 where it exits the chamber outlet into valve 111. Valve 111 controls the flow of gas to valve 116 which flows into chamber 110 such that the pressure in 108 can be controlled, varied, or oscillated in conjunction with valve 112. Chamber 110 contains ethanol for example but other aforementioned solvents and mixtures can be used. As the plant material is heated under gas flow to the desired temperature range between 150° C. to 350° C. by heating element 109, volatile terpenoids and flavonoids are scrubbed and captured in the liquid mixture contained in chamber 110. As the gas flow through chamber 110, it passes through the chamber outlet into valve 113 where it is vented to the atmosphere. Alternatively, the gas can be returned to reservoir 107 to recycle and keep the gas in a closed loop. Once thermal treatment of the plant material is complete, the heat is reduced by element 109 (optionally integrated cooling). The gas flow is then diverted by valve 112 and passes to valve 113 wherein valve 113 diverts the flow though the chamber 110, effectively pressurizing the chamber 110. Valve 116 diverts the pressurized flow of the liquid mixture and gas into chamber 108 through 111 where thermally treated plant material is contained. The liquid mixture is allowed to extract the plant material at the desired pressure, temperature, and time. Next, the pressurized gas is directed by valve 112 flows into chamber 108 where in valve 111 and valve 116 move the liquid extract mixture to chamber 110 wherein the heating/cooling element 115 is set to the desired temperature to vaporize the solvent. The vaporized solvent then passes through valve 113 and is directed into condensing chamber 117 wherein the vapor is converted back to a liquid suitable for subsequent processing.

Pressurized gas from 107 is directed by valves 112 and 113 to push the finished composition through valve 116 which directs the composition to chamber 114 wherein chamber 114 can be, but is not limited to, a finished goods package. It is understood that further optimization of valve controls, temperature controls, pressure controls and other automation is possible to increase process efficiency. In addition, the method of preparation may be performed at large scale in a factory environment or may be performed on a small, individualized scale at the point-of-sale where individual customer orders may be customized according to user preferences.

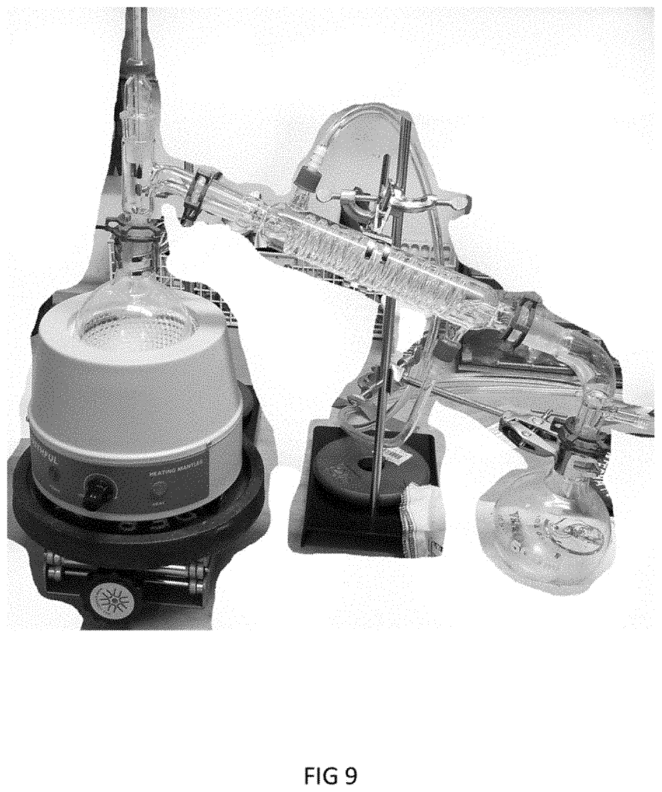

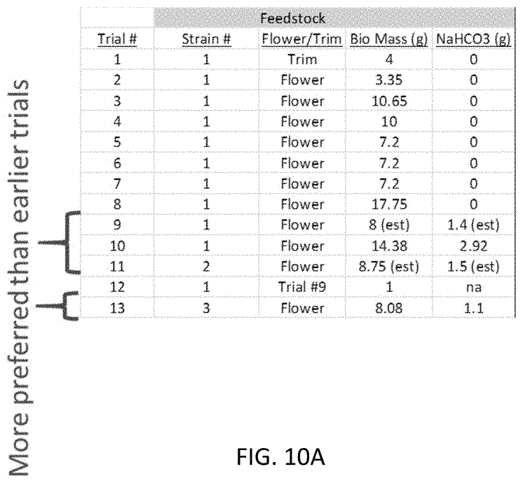

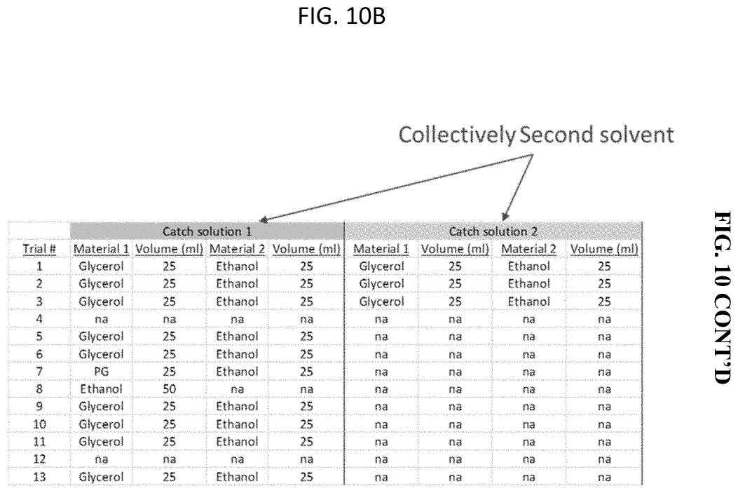

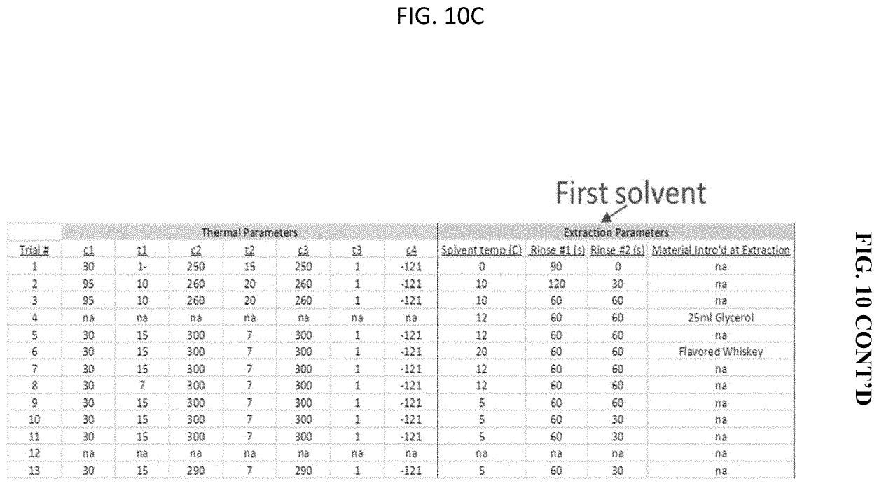

A bench scale process was built to produce compositions derived from cannabis in accordance to the disclosed disclosure. Shown in FIG. 8, an MTI tube furnace, model OTF-1200X-S-NT-50-110-2” 1250° C. Compact Split Tube Furnace with 8″ Long heating Zone & 30 segment Controller, equipped with a glass tube, 500Dx44 IDx 450 L, mm (2″D×17.7″L) fitted with stainless steel endcaps with tubing connectors. Ultra-high purity grade nitrogen was connected to the inlet and outlet ports of the glass tube using food grade silicone tubing. In addition, the outlet tubing was connected to two sequentially arranged 125 mL Drecshel's Bottles (Interchangeable Joint Head 24/29, Borosilicate 3.3 Glass) containing glass beads and a mixture of 200 proof ethanol and vegetable glycerin. The outlet of the Drecshel bottle was vented to atmosphere. In addition, shown in FIG. 9, a complete distillation setup (250 ml 3930-55), including a heating mantel suitable for round bottom flasks was used for all distillation processing. A chiller unit capable of reaching 0° C. was used to supply the condensers with circulating water. An analytical balance was used for all weight measurements and standard supplemental lab supplies were used in process work. Sample compositions were made according to the parameters shown in FIG. 10, placed in sample vials under nitrogen and kept in refrigerated storage. Feed stock for all experiments consisted of various types of cannabis flower and other plant material as designated. The range of processing conditions, such as thermal temperature, time at temperature, quantity of plant material processed, Deschle bottle compositions used to entrain volatile organic species, extraction time, extraction temperature, plant material type, and other processing conditions are disclosed in Tables 1-3 as non-limiting parameters.

In general, cannabis flower was manually ground using a standard hand grinder to reduce the particle size of the plant material.

A predetermined quantity of plant material was placed on a stainless-steel perforated sample stage and inserted into the furnace tube such that it was centered in the heating region of the furnace. Once the endcaps were secured, the UHP nitrogen was introduced at a sufficient rate to essentially purge and remove oxygen in the sealed glass furnace tube. Nitrogen passed from the tube furnace outlet into the Dreshel bottles containing equal amounts of 200 proof ethanol and vegetable glycerin according to Table 2. The system was allowed to purge for approximately 10 minutes prior to initiating the heating cycle to the set temperature. Once the temperature program was complete and the furnace had cooled to at least 35° C., the sample of thermally treated plant material was removed and immediately placed in a container with about 50 ml of 200 proof ethanol, typically ranging between 0° C. and 15° C. The plant material was extracted for a predetermined amount of time at room temperature. Once the plant material was filtered from the extract, the resultant extract was combined with the contents of the Dreshle bottle in a 500 ml round bottom flask. The flask was connected to the distillation setup wherein a standard technique was used to distill the ethanol from the composition. The resulting composition was transferred to 25 ml sample vials under nitrogen for refrigerated storage. It is understood that that the conditions described herein are non-limiting examples and served to demonstrate the utility of the disclosure. Control samples were also made by extracting equivalent non-thermally treated plant material using 200 proof ethanol and subsequently combining the extract with fresh Dreshle bottle solution that was not used to capture entrained volatile compounds. Finally, the control was distilled in the same manner as thermally treated plant material to remove the ethanol. Compositions were suitable for use in electronic cigarettes for organoleptic evaluation. Compositions were evaluated by users familiar with electronic cigarette devices and a range of experience with cannabis products. The following sections describe user response and perception of compositions produced according to the disclosure.

Control compositions visually appeared to be light green translucent to turbid in color with evidence of some insoluble low-density material that floated to the sample surface. In addition, those samples had a strong and unpleasant terpene odor. In stark contrast, compositions according to the disclosure have a dark brown color and no green terpenoid odor. It is also noted that insoluble low-density material floated to the composition surface. This is presumably due to the lack of solubility in vegetable-based glycerin. Furthermore, use of propylene glycol in the Dreshle bottle instead of glycerin in trial #7 appeared to dramatically reduce the amount of insoluble material, indicating that the selection of propylene glycol to reduce insoluble material is an appropriate choice to solubilize the unique chemistry of the compositions made according to the disclosure. Solvents can be selected based on, but not limited to, polarity, pH, pKa, pKb, hydrophilicity, hydrophobicity, other acidic or basic parameters, solubility parameters, vapor pressure, Hildebrand solubility parameters, and the like singularly or in any combination. The processed products of trials 9-26 were preferred over the earlier trails (see Table 1). Catch solutions #1 and #2 (see Table 2) collectively make up the second solvent/solvent system.

TABLE 1

| 9 | 8 | 7 | 6 | 5 | 4 | 3 | 2 | 1 | Trial # | |
|---|---|---|---|---|---|---|---|---|---|---|
| Jun-19 | May-19 | May-19 | May-19 | May-19 | May-19 | May-19 | May-19 | May-19 | | Date |
| Same as 5 | Extract | Same | Same | Aggressive | Baseline | Triple | First | First | | Intent of |
| but with | only | as 5 | as 5 | double | (Unprocesse | concentrate | trial of | trial of | | Trial |
| baking | into | but with | but with | platform | d extract) | the catch | flower | trim | | |
| soda | ethanol | PG | Whiskey | roast | | solution | | | | |
| Phantom | Phantom | Phantom | Phantom | Phantom | Phantom | Phantom | Phantom | na | Strain Name | Feedstock |
| Land | Land | Land | Land | Land | Land | Land | Land | | | |
| 1 | 1 | 1 | 1 | 1 | 1 | 1 | 1 | 1 | Strain # | |
| Outdoor | Outdoor | Outdoor | Outdoor | Outdoor | Outdoor | Outdoor | Outdoor | Outdoor | Indoor/Outdoor | |
| Flower | Flower | Flower | Flower | Flower | Flower | Flower | Flower | Trim | Flower/Trim | |
| 8 (est) | 17.75 | 7.2 | 7.2 | 7.2 | 10 | 10.65 | 3.35 | 4 | Bio Mass (a) | |
| 1.4 | 0 | 0 | 0 | 0 | 0 | 0 | 0 | 0 | NaHCO3 (g) | |

| 20 | 19 | 18 | 17 | 16 | 15 |
|---|---|---|---|---|---|
| May 18, 2020 | May 2, 2020 | Apr. 28, 2020 | Apr. 26, 2020 | Jan-20 | Jan-20 |
| Sample 13 | Sample 13 | Sample 10 | Same as 9 | Hemp CBD | Hemp CBG |
| blended 3 | blended 1:1 | filtered | but into | Flower | Flower |
| parts to 1 | ratio with an | through | MCT oil | 70/30 | 80/20 |
| part Gorilla | oil | cheesecloth | | VG/PG w/ | VG/PG |
| Glue | | | | higer | |
| | | | | concentratio | |
| | | | | n | |
| Phantom | Phantom | Phantom | Phantom | Hawaiian | White CBG |
| Land | Land | Land | Land | Haze | |
| 1 | 1 | 1 | 1 | 4 | 5 |
| Outdoor | Outdoor | Outdoor | Outdoor | Outdoor | Outdoor |
| Flower | Flower | Flower | Flower | Flower | Flower |
| | | | 17.96 | 8.5423 | 9.42 |
| | | | 2.8 | 1.1134 | 1.04 |

| 14 | 13 | 12 | 11 | 10 | |
|---|---|---|---|---|---|
| Jan-20 | Jun-19 | Jun-19 | Jun-19 | Jun-19 | Date |
| Hemp CBD | Same as 9, | Blend | Same as 9, | Same as 9, | Intent of |
| Flower | but with | externally | but with | but with 4 | Trial |
| 80/20 | different | purchased | different | platforms to | |
| VG/PG | feedstock, | cannabinoid | feedstock | increase | |
| | lower temp | oil with trial | | concentratio | |
| | | #9 | | n | |
| Hawaiian | Blueberry | n/a | CBD Shark | Phantom | Feedstock |
| Haze | Muffin | | | Land | |
| 4 | 3 | 1 | 2 | 1 | |
| Outdoor | Indoor | na | Outdoor | Outdoor | |
| Flower | Flower | Trial #9 | Flower | Flower | |
| 8.15 | 8.08 | 1 | 8.75 | 14.38 | |
| 1.02 | 1.1 | na | 1.5 | 2.92 | |

| 26 | 25 | 24 | 23 | 22 | 21 | |
|---|---|---|---|---|---|---|
| Jul. 11, 2020 | May 27, 2020 | May 27, 2020 | May 18, 2020 | May 18, 2020 | May 18, 2020 | Date |
| Hemp CBD | Sample 15 | Sample 15 | Sample 14 | Sample 13 | Sample 13 | Intent of |
| bitters with | in Aai | with 1% Nic | in | in cart from | in | Trial |
| 18% | disposable | Salt | disposable | Shenzhen | disposable | |
| retained | | (Benzoic) in | | ECAP | from AAI | |
| alcohol | | vial | | | | |
| Hawaiian | White CBG | White CBG | Hawaiian | Phantom | Phantom | Feedstock |
| Haze | | | Haze | Land | Land | |
| 4 | 5 | 5 | 4 | 1 | 1 | |
| Outdoor | Outdoor | Outdoor | Outdoor | Outdoor | Outdoor | |
| Flower | Flower | Flower | Flower | Flower | Flower | |
| 8.34 | | | | | | |
| 1.25 | | | | | | |

TABLE 2

| 9 | 8 | 7 | 6 | 5 | 4 | 3 | 2 | 1 | Trial # | |
|---|---|---|---|---|---|---|---|---|---|---|
| Glycerol | Ethanol | PG | Glycerol | Glycerol | na | Glycerol | Glycerol | Glycerol | Material 1 | Catch |
| 25 | 50 | 25 | 25 | 25 | na | 25 | 25 | 25 | Volume (ml) | solution |
| Ethanol | na | Ethanol | Ethanol | Ethanol | na | Ethanol | Ethanol | Ethanol | Material 2 | vessel 1 |
| 25 | na | 25 | 25 | 25 | na | 25 | 25 | 25 | Volume (ml) | (Second |
| | | | | | | | | | | Solvent |
| | | | | | | | | | | Part 1) |
| na | na | na | na | na | na | Glycerol | Glycerol | Glycerol | Material 1 | Catch |
| na | na | na | na | na | na | 25 | 25 | 25 | Volume (ml) | solution |
| na | na | na | na | na | na | Ethanol | Ethanol | Ethanol | Material 2 | vessel 2 |
| na | na | na | na | na | na | 25 | 25 | 25 | Volume (ml) | (Second |
| | | | | | | | | | | solvent |
| | | | | | | | | | | part 2 (if |
| | | | | | | | | | | used)) |

| 21 | 20 | 19 | 18 | 17 | 16 | 15 | 14 | 13 | 12 | 11 | 10 |
|---|---|---|---|---|---|---|---|---|---|---|---|
| | | | | MCT | Glycerol | Glycerol | Glycerol | Glycerol | na | Glycerol | Glycerol |
| | | | | 50 | 10 | 20 | 20 | 25 | na | 25 | 25 |
| | | | | Ethanol | Ethanol | Ethanol | Ethanol | Ethanol | na | Ethanol | Ethanol |
| | | | | 50 | 35 | 20 | 20 | 25 | na | 25 | 25 |
| | | | | na | PG | PG | PG | na | na | na | na |
| | | | | na | 5 | 5 | 5 | na | na | na | na |
| | | | | na | Ethanol | Ethanol | Ethanol | na | na | na | na |
| | | | | na | 0 | 5 | 5 | na | na | na | na |

| 26 | 25 | 24 | 23 | 22 |
|---|---|---|---|---|
| Glycerol | | | | |
| 25 | | | | |
| Ethanol | | | | |
| 25 | | | | |
| na | | | | |
| na | | | | |
| na | | | | |
| na | | | | |

TABLE 3

| 3 | 2 | 1 | Trial # | |
|---|---|---|---|---|
| 95 | 95 | 30 | Initial temp | Thermal |
| 10 | 10 | 1- | Ramp up time | Parameters |
| | | | (minutes) | |
| 260 | 260 | 250 | Temp 1 | |
| 20 | 20 | 15 | Hold time | |
| | | | (minutes) | |
| 260 | 260 | 250 | Temp 2 | |
| 1 | 1 | 1 | Ramp down | |
| | | | time (minutes) | |
| −121 | −121 | −121 | End temp | |
| | | | (anything less | |
| | | | than 0 = OFF) | |
| 10 | 10 | 0 | Solvent temp | Collection |
| | | | (C.) | Parameters |
| Ethanol | Ethanol | Ethanol | Solvent | (using First |
| 60 | 120 | 90 | Rinse #1 (s) | Solvent) |
| 60 | 30 | 0 | Rinse #2 (s) | |
| na | na | na | Additional | |
| | | | Material Intro'd | |
| | | | during | |
| | | | Extraction | |

| 14 | 13 | 12 | 11 | 10 | 9 | 8 | 7 | 6 | 5 | 4 | |
|---|---|---|---|---|---|---|---|---|---|---|---|
| 30 | 30 | na | 30 | 30 | 30 | 30 | 30 | 30 | 30 | na | Thermal |
| 15 | 15 | na | 15 | 15 | 15 | 7 | 15 | 15 | 15 | na | Parameters |
| 290 | 290 | na | 300 | 300 | 300 | 300 | 300 | 300 | 300 | na | |
| 7 | 7 | na | 7 | 7 | 7 | 7 | 7 | 7 | 7 | na | |
| 290 | 290 | na | 300 | 300 | 300 | 300 | 300 | 300 | 300 | na | |
| 1 | 1 | na | 1 | 1 | 1 | 1 | 1 | 1 | 1 | na | |
| −121 | −121 | na | −121 | −121 | −121 | −121 | −121 | −121 | −121 | na | |
| 0 | 5 | na | 5 | 5 | 5 | 12 | 12 | 20 | 12 | 12 | Collection |
| Ethanol | Ethanol | na | Ethanol | Ethanol | Ethanol | Ethanol | Ethanol | Ethanol | Ethanol | Ethanol | Parameters |
| 30 | 60 | na | 60 | 60 | 60 | 60 | 60 | 60 | 60 | 60 | (using First |
| 30 | 30 | na | 30 | 30 | 60 | 60 | 60 | 60 | 60 | 60 | Solvent) |
| na | na | na | na | na | na | na | na | Flavored | na | 50 ml | |
| | | | | | | | | Whiskey | | Glycerol | |

| 25 | 24 | 23 | 22 | 21 | 20 | 19 | 18 | 17 | 16 | 15 | |
|---|---|---|---|---|---|---|---|---|---|---|---|
| | | | | | | | | 30 | 30 | 30 | Thermal |
| | | | | | | | | 15 | 15 | 15 | Parameters |
| | | | | | | | | 290 | 290 | 290 | |
| | | | | | | | | 7 | 7 | 7 | |
| | | | | | | | | 290 | 290 | 290 | |
| | | | | | | | | 1 | 1 | 1 | |
| | | | | | | | | −121 | −121 | −121 | |
| | | | | | | | | 0 | 0 | 0 | Collection |
| | | | | | | | | Ethanol | Ethanol | Ethanol | Parameters |
| | | | | | | | | 30 | 30 | 30 | (using First |
| | | | | | | | | 30 | 30 | 30 | Solvent) |
| | | | | | | | | na | na | na | |

| | |
|---|---|
| | Collection Parameters |
| Thermal Parameters | (using First Solvent) |

| | | | | | | | | | | | |
|---|---|---|---|---|---|---|---|---|---|---|---|
| 26 | 30 | 15 | 280 | 7 | 280 | 1 | −121 | 0 | 15 | 15 | na |

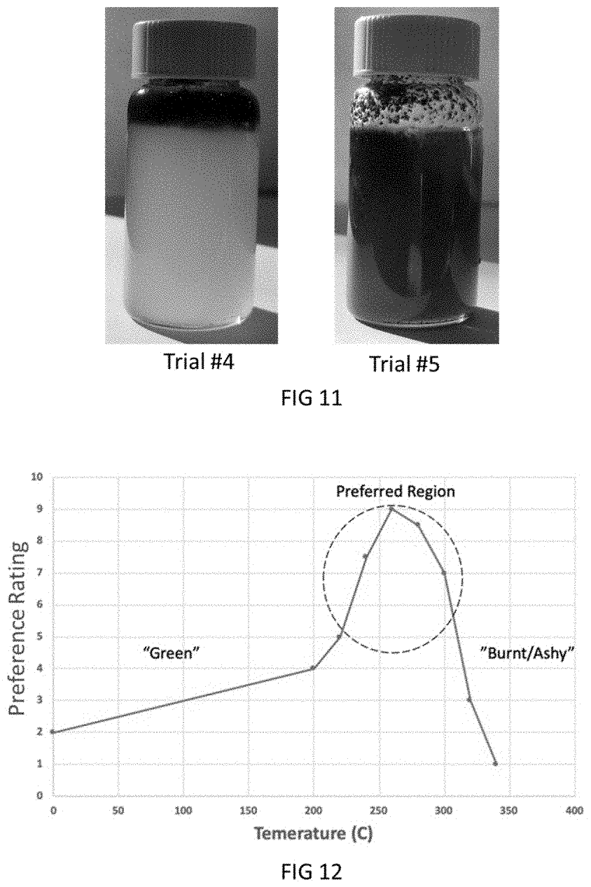

FIG. 11 illustrates the difference in visual appearance of typical control compositions (trial #4) compared to compositions prepared according to the disclosure (trial #5). It is noted that the control samples have a similar appearance to products available in the market place. The unexpected, striking difference in color of the compositions compared to the control is due to the unique chemical profile of the compositions prepared according the disclosure versus the chemical profile of compositions that are not prepared according to the disclosure. It is reasonable to expect that a distinct, unique chemical composition of THC, CBD, other cannabinoids, flavonoids, terpenoids and other desirable chemistries and mass ratios thereof in any combination or in part, are associated with compositions made according to the disclosure. It is reasonable to expect standard analytical methodologies such as, but not limited to, mass spectrometer, NMR, gas chromatography, high performance liquid chromatography, Infrared spectroscopy (FTIR) and the like can be used, singularly or in any combination, to determine the unique chemical profile, quantitative ratios and compositional chemistries, that further define desirable compositions according to the disclosure described herein. It is anticipated that for each processing condition a commensurate, unique chemical fingerprint exists that is only possible by practicing the disclosure herein.

Another surprising feature of the compositions according to the disclosure is the apparent stability during refrigeration. The compositions essentially remained the same as they were first processed.

As the compositions were suitable for vaping using an electronic cigarette device, the organoleptic properties of the compositions were evaluated as a non-limiting method of comparative assessment. Individuals having a range of experience with vaping devices were selected to evaluate the compositions from Table 2. Compositions were placed in a refillable cartridge of a commercially available electronic cigarette. Compositions were evaluated in vaping sessions lasting 5 to 10 minutes wherein preference was ranked on a 10-point scale with 10 representing the maximum value. A graphical representation of the evaluation is shown in FIG. 12 wherein preference versus processing temperature for a predetermined time is plotted.

Compositions produced below about 200° C. were deemed to have a lower preference and described as having a green or terpene character. The unfavorable thermal region is denoted as “Green” region in FIG. 8. In addition, thermal treatments substantially above about 300° C. tended to be described as having an ashy or burnt characteristic and also scored lower in preference. Surprisingly, compositions that scored the highest in preference were between about 190° C. to about 315° C. as designated in FIG. 12 estimated by the preferred region. It is understood that the temperature ranges identified are non-limiting examples of preferred processing conditions. It is also further understood that different plant material, moisture levels of the plant material, extraction rates, extraction temperatures, extraction medium, pressure of processing conditions for example will have preferred processing temperature conditions.

Yet another embodiment involves supplying additives to the plant material prior to, during, or combinations thereof, thermal treatment (i.e., heating) process. These additives further enable the disclosure described herein. It has been unexpectedly discovered that acidic or basic organic and/or inorganic chemistries added to the plant material resulted in highly desirable, unique organoleptic characteristics of the compositions according to the disclosure. Additives in the form of solids, liquids, gases, or combinations thereof can be introduced to thermal process during any point of thermal cycle. Acids or bases can be selected based on criteria such as, but not limited to, pka, pkb, pH at a specific aqueous concentration, mono protic, poly protic, mono basic, or polybasic, inorganic or organic, water solubility, hydrophobicity, hydrophilicity vapor pressure at defined temperatures, melting point, sublimation point, supercritical point, and the like. Not bound by theory, it is reasonable that different specific plant constituents react, or participate in reactions with, but not limited to, each other, thermal rearrangements, thermal decomposition, sub stoichiometric oxidation reactions and the like. Furthermore, it has been found that these reactions are only possible under the conditions described herein and result in chemical compositions/extracts with extraordinary taste, smell and consumability. Surprisingly, it appears that thermal treatment (i.e., heating) under an essentially oxygen free atmosphere at temperatures above 150° C. facilitates the aforementioned reaction types. Furthermore, the use of the afore mentioned acidic or basic species can further enhance and/or augment desirable reactions between sugars and proteins (amino acids) found in the plant material, or alternatively added, during thermal treatment (i.e., heating) process.

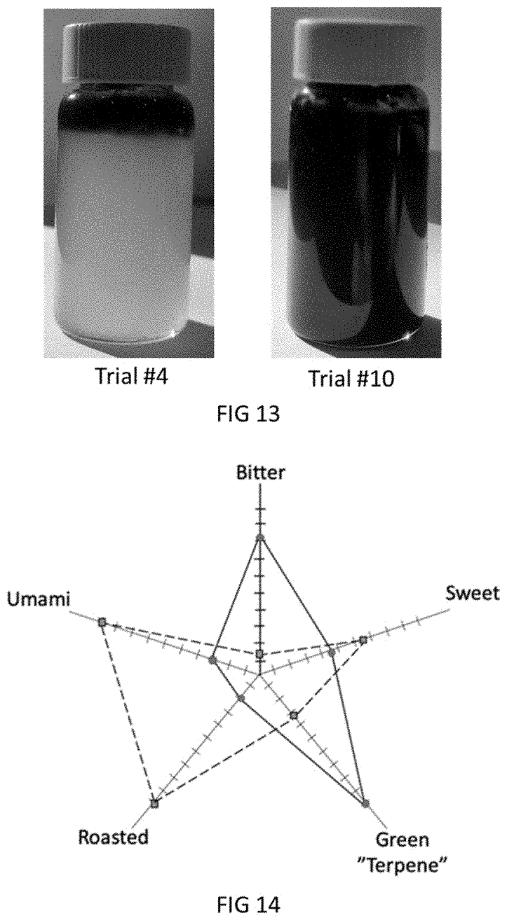

It has surprisingly been discovered that reactions such as, but not limited to, the Maillard reaction are only possible under the conditions described herein. As such the compositions have superior organoleptic characteristics over compositions and extracts that do not use the disclosure disclosed herein. It is believed, and not bound by theory, that for example, carbohydrates (sugars) and proteins can react to form glycosamines wherein the glycosamines isomerize by the Amadori rearrangement to produce ketosamines can further react by different mechanism to produce flavaniods and other favorable organoleptic chemistries such as fission products and reductones as a result of basic conditions, and hydroxymethylfurferal as result of acidic conditions. Furthermore, these reactions produce, but are not limited to, compounds such as pyrazines, pyrroles, acrylpyridines, furanones, furans, and oxazoles, all of which have desirable smell and taste. Surprisingly, the use of food grade sodium carbonate added to the plant material in approximately 1:8 weight ratio of sodium carbonate to plant material was sufficient to significantly impact the extent and rate of the afore mentioned reactions. Compositions made with the addition of the mild base, sodium carbonate, had much deeper color, stronger more pleasant smell, and the finished liquid appeared completely uniform. FIG. 13 illustrates the dramatic difference in appearance of the control composition and compositions made according to the disclosure. The reaction chemistry rendered the previously insoluble materials highly soluble in vegetable-based glycerin, even more so than trial #5 which used no sodium bicarbonate. Moreover, the compositions made using mild base additives were unexpectedly stable during storage under refrigerated conditions. This is an unpredicted benefit of using mild based additives, and based on the same principle, one can also use acidic additives. Additionally, no winterizing is necessary to produce homogenous compositions. The advantage of avoiding winterizing processes is the ability to retain desirable plant-based elements in compositions that would otherwise be lost or albescent in final compositions. In this regard, compositions made according to the disclosure are superior to current known processing methods and associated products.

As the dramatic difference in appearance is a direct result of the unique chemistry produced during the aforementioned processing step according to the disclosure, it is reasonable to expect quantitative delineation of chemical nature of composition Trial #10. Again, the previously mentioned analytical analysis techniques can be used to quantitate and define the unique chemistries responsible for the desirable organoleptic and physical properties of compositions made with acids or bases added to the plant material according to the disclosure. Furthermore, qualitative evidence of the favorable reactions are illustrated by user ranking of taste attributes for the two compositions made with same cannabis plant material: one made using the disclosure (FIG. 10), including sodium carbonate additive, and the other as a control (Trial #4) that did not use thermal process of the disclosed disclosure. Again, compositions were placed in refillable electronic cigarette cartridges and attached to appropriate power modules suitable for vaping. Users were given both control and test composition to evaluate and rank according to their perception of the organoleptic characteristics based on bitter, sweet, green/terpene, roasted, umami taste dimensions on a 10-point scale where 10 represents the maximum ranking of a particular dimension.

FIG. 14 represents the comparative rankings in the different organoleptic dimensions on a spider diagram. Compositions made according to the disclosure were vastly superior to the control compositions made in a similar manner but not thermally processes according to the disclosure. The preferred composition had superior taste characteristics with regard to having no overpowering “terpene” or green taste, less bitterness, highly desirable umami taste, and roasted flavor not experienced in current known available products. In strong contrast, the control composition was characterized by a green/terpene taste, less sweet, and essentially devoid of roasted or umami character. It appears that the unique chemistry of the compositions as a result of processing according to the disclosure gives rise to the desirable organoleptic characteristics.

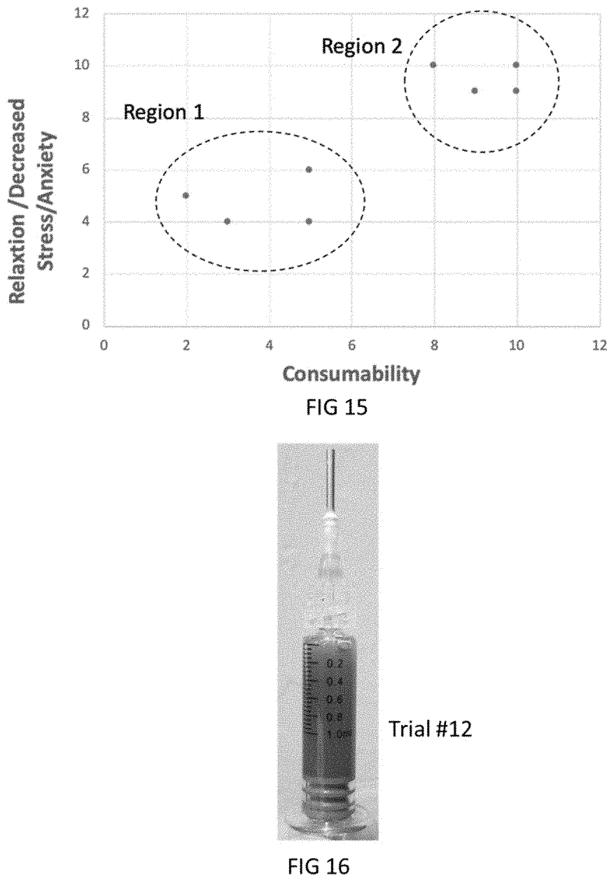

In addition, a relevant metric to evaluate the utility of the compositions based on their unique chemistry is consumability. That is to say, the ability to consume or use compositions for an extended period of time between 30 minutes and 2 hours without having sensor saturation or experience impairment during consumption. Users were asked to continue to use samples as described in FIG. 15 for consumability, extended use, and rank the usability on a 10-point scale, where a rating of 10 represents maximum favorable perception. In addition, another useful dimension to evaluate the utility of the compositions the reduction of stress or anxiety and a general feeling of well-being, happiness and relaxation. This too was determined for the same samples reference above on a 10-point scale were 10 represents the maximum effect experienced. FIG. 15 demonstrates the relationship between consumability and reduction in stress/anxiety and further demonstrates therapeutic effect of the compositions according to the disclosure.

Control compositions did not appear to have a sustained consumability compared to the compositions made in accordance with the disclosure. In FIG. 15, Region 1 depicts the perceived consumability and resulting feeling of relaxation and decreased stress/anxiety in the control composition. In contrast, compositions made according to the disclosure had relatively high scores for consumability and ability to impart a feeling of reduced stress/anxiety with an overarching feeling of relaxation as depicted by Region 2. Surprisingly the effectiveness of composition made according to the disclosure are superior to compositions that do not take advantage of the disclosure. In addition, the preferred compositions possess the organoleptic characteristics and therapeutic effects that overcome the limitations of current product offerings based on cannabis extracts.

Compositions made according to the disclosure can be consumed as-is. Additionally, compositions that result from thermal treatment (i.e., heating) process may be combined with other compositions in order to impart the preferred organoleptic properties to the combined compositions. FIG. 16 shows Trial #12, a 50/50 blend of a Trial #9 and a cannabis oil that was manufactured via super-critical CO2 extraction process. The combined composition had superior taste and smoothness characteristics to the cannabis oil alone. The combined composition also had higher cannabinoid content than Trial #9 alone. The ratio of composition subjected to thermal treatment (i.e., heating) process to composition from other processes can be varied in order to create combined compositions with a range of potencies and organoleptic properties. Such compositions can be designed to be inhaled by the user while other compositions can be designed to be ingested by the user.

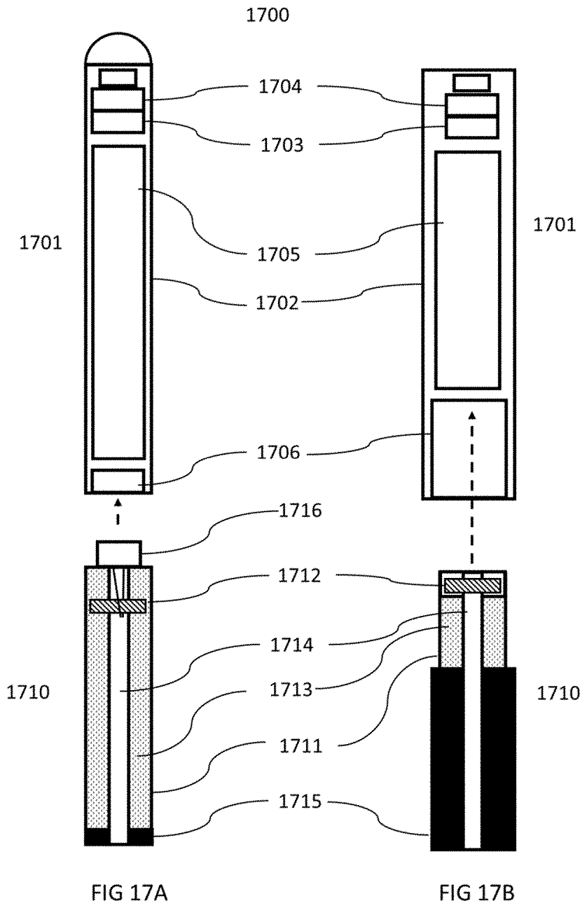

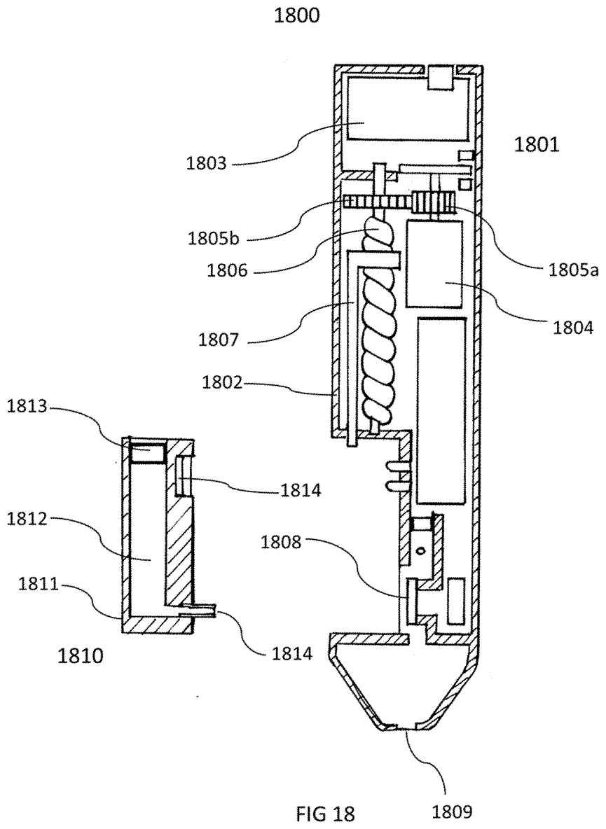

FIGS. 17A and 17B describe a passive vaporizer article 1700 comprised of a passive control device 1701 and passive inhalation media cartridge 1710 that can be used in conjunction with compositions made according to the disclosure. The passive vaporizer article 1700 can be commonly referred to as an ecig and is the basic design used by well-known products, including, but not limited to: blu ecigs plus+, blu ecigs my blu, RJ Reynolds Vuse, RJ Reynolds Solo, and JUUL. The passive inhalation media cartridge 1710 is further comprised of a passive cartridge housing 1711 and an internal air tube 1714 located within the passive cartridge housing 1711. The space between the passive cartridge housing 1711 and internal air tube 1714 forms a passive inhalation media storage area 1713 for the storage of inhalation media which can be comprised of a thermally processed composition according to the present disclosure. The passive cartridge housing 1711 can be a circular tube, like blue cigs plus+, or can have a non-circular cross section, like the JUUL cartridge. The internal air tube 1714 can be circular tube or can have a non-circular cross section. The passive inhalation media storage area 1713 can be bounded at one end by the passive mouthpiece 1715, compliant plug (not shown), or an internal wall feature (not shown). The passive media storage area 1713 can be bounded at the opposite end by the passive cartridge connector 1716 or other component (not shown). A passive wick and heater 1712 is located within the passive cartridge housing 1711 and is in fluid communication with the inhalation media. A portion of the passive wick and heater 1712 can pass through holes formed in the internal air tube 1714, passive cartridge connector 1716, heater chamber, or other internal component in order to transport inhalation media from the passive inhalation media storage area 1713 to the heater portion of the passive wick and heater 1712 where it can be vaporized and entrained in the air stream for deliver to the user via the internal air tube 1714 and passive mouthpiece 1715. The passive wick and heater 1712 may be constructed from a non-electrically conductive porous material that transports the inhalation media via capillary action and a resistance wire. Such porous materials can include, but are not limited to: porous ceramic, non-conductive treated metal mesh, cotton, stranded or woven silica or other similar material. Alternatively, the passive wick and heater 1712 can be situated within additional internal components other than the internal air tube 1714, for example, an internal vaporization chamber that is fluidly connected with the internal air tube 1714. The resistance wire can also be constructed to reside within the porous material, for example, it can be positioned within porous ceramic material before the ceramic material is hardened during manufacturing.

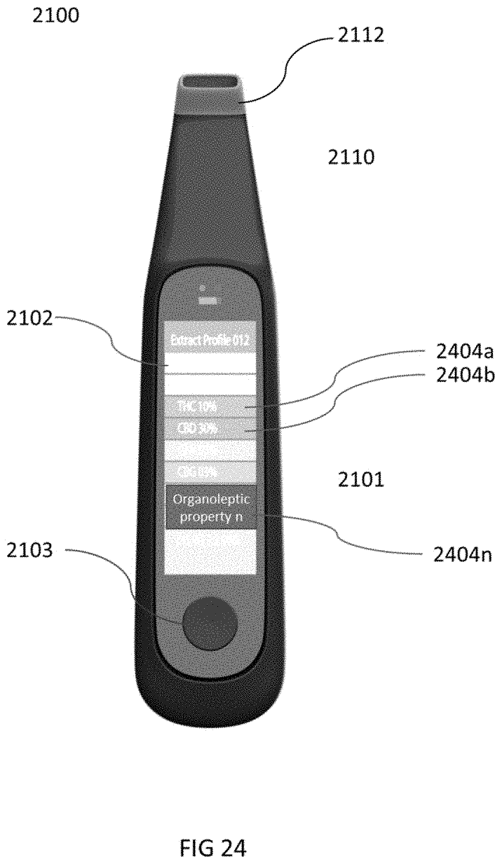

The passive cartridge connector 1716 can provide electrical connections between the passive control device 1701 and the resistance wire of the passive wick and heater 1712. The passive cartridge connector 1716 can be shaped so as to mechanically and electrically connect with the passive control device connector 1701. Mechanical retention features such as screw threads, locking gaps, locking detents, snap fits, friction fits, and magnets can be provided on one or both the passive cartridge connector 1716 and passive control device connector 1706 in order to ensure robust mechanical and electrical connections. Some versions of passive vaporizer article 1700 eliminate the need for a passive cartridge connector 1716 by having all or a portion of the passive inhalation media cartridge 1710 nest within the passive control device 1701 for mechanical alignment and retention, shown in FIG. 24b. Such systems are commonly be referred to as “pod” systems.

The passive control device 1701 can be comprised of a passive control device housing 1702, battery 1705, inhalation sensor 1704, passive control circuit 1703, and one or more indicator lights. The passive control circuit 1703 can be configured to provide electrical energy to the resistance wire or the passive wick and heater 1712. The inhalation sensor 1704 can be configured to detect either of a pressure change or change in air flow rate caused by the inhalation of the user. The inhalation sensor may alternatively be replaced with a button or switch that may be activated manually by the user. The passive control device 1701 can be configured to perform many of the functions, including, but not limited to: connecting to a computer network 1801, recording doses, sending and receiving dosing information, exchanging data with a computing device 1803, dose compensation, remote dosing, and controlling the temperature of the resistance wire.

FIG. 18 shows section views of a control device 1801 and cartridge 1810 that are well suited to deliver doses of inhalation media comprising thermally processed compositions. Because wick-based systems can act to preferentially filter components of the inhalation media and can have undesirable performance, a non-wick-based system can be used to provide optimal delivery of compositions created according to the disclosure. The control device 1801 contains a control circuit 1803 that governs the operation of the control device 1801. The control circuit 1803 is connected to a charge connector for the purpose of receiving power and charging a battery. The control circuit 1803 can be configured to accept input from a variety of input controls, including, but not limited to; buttons, a selector dial, or control pad 130. The control circuit 1803 can govern the operation of indicator lights and may also be configured to drive a display screen. When the control circuit 1803 receives a dosing command, it determines the drive signal needed to express the desired about of inhalation media. The inhalation media is expressed via the action of the plunger driver 1807 moving the plunger 1813 a determined distance in order to change the volume of the inhalation media storage area 1812 by a determined amount and thusly force a precise amount of inhalation media through the cartridge outlet 1814. This can be a more precise way to transport inhalation media to the heater without filtering the inhalation media through a wicking material. When the cartridge 1810 is mated with the control device 1801, the plunger driver 1807 is aligned with the plunger 1813. The plunger driver 1801 can have a female threaded portion that is connected to a drive screw 1806. In the embodiment shown in FIG. 18, the plunger driver 1807 is not co-axial with the drive screw 1806, however, alternative embodiments of the plunger driver 1807 consist of a co-axial configuration where the plunger driver 1807 fully encompasses all but one end of the drive screw 1806. In the embodiment shown in FIG. 18, the drive screw 1806 is constrained by the housing 1802 in all degrees of freedom except rotation about its major axis. The drive screw 1806 is rigidly connected to a driven gear 1805b and can alternatively be integrally formed with the driven gear 1805b. Driven gear 1805b is driven by drive gear 1805a which in turn is rigidly connector to the shaft of the dispensing motor 1804. It should be noted that drive gear 1805a could be replaced with a series of gears or gearhead in order to achieve the necessary torque to drive the system. This torque amplification through gearing scheme is employed in order to be able to use a small motor while still deliver sufficient drive force at the plunger driver 1807. If size constraints were not a concern, or hi-torque motors became available, the gears could be eliminated and the plunger driver 1807 could be driven directly from dispensing motor 1804.

An optical encoder disk is connected to the shaft of dispensing motor 1804. The optical encoder disk can be connected to the drive gear 1805a side of the dispensing motor 1804 or can be connected on the opposite end of the dispensing motor 1804, provided that a portion of the motor shaft extends out past the non-gear side of the housing of the dispensing motor 1804. In other embodiments, the optical encoder disk 118 can be mounted to the drive screw 1806 or a gear, however, mounting the optical encoder disk to the motor shaft is preferred because it provides higher resolution determination of the position of the plunger driver 1807 due to the gear ratio between the drive gear 1805a and the plunger driver 1807. The optical encoder disk is comprised of a series of equally spaced and sized openings that allows light from an emitter to be received by a photo detector when the opening is aligned with the emitter and light from the emitter to be blocked when the opening is not aligned. As the motor shaft rotates, the photo detector will produce a digital signal where the rate of the digital signal transitions corresponds to the rotational rate of the motor shaft. Since the emitter and photo detector are electrically connected to the control circuit 1803, the control circuit 1803 can use this rate information to calculate how much the motor has turned and thus, how far the plunger driver 1807 has traveled. Alternatively, the control circuit 1803 can be configured to use position information rather than rate information from the photo detector in order to determine how far the plunger driver 1807 has traveled. The control circuit 1803 having determined how far to drive the plunger driver 1807 in order to deliver a requested amount of inhalation media, and controlling the motor to achieve this travel, the precise dose is dispensed onto the vaporizer element 1808.

The preceding description describes a system that uses a particular style of drive train and particular style of sensor in order to achieve the dosing function. Alternative approaches can be used to achieve this function. For example, the dispensing motor 1804 can be a stepper motor whereby the drive signal determines an incremental advancement of the motor shaft, thus eliminating the need for the optical encoder disk, emitter and photo detector. Alternatively, the dispensing motor 1804 can be a linear motor. In this case, the linear motor could be directly connected to the plunger driver 1807 or be connected via a linear drivetrain. Also, rather than a gear style drivetrain, the system could employ linkages, belts and pulleys or cable drives to connect the plunger driver 1807 to the dispensing motor 1804. The optical encoder disk, emitter and photo detector can be replaced by a hall effect sensor where the hall effect magnet portion takes the place of the optical encoder disk and is mounted to a portion of the motor shaft. The hall effect magnet can have one or more sets of magnetic poles. In this case, one or more hall effect sensors, which replace the photo detector can be mounted to a stationary portion of the dispensing motor 1804, a stationary PCB or a stationary portion of the housing 1802 and be configured to detect the rotation of the hall effect magnet. Alternatively, optical encoder disk, emitter, and photo detector can be replaced by a potentiometer attached to a portion of the motor shaft. Alternatively, an optical linear encoder, linear hall effect sensor or linear potentiometer may be mounted to or integrally formed with the plunger driver 1807.

After a dose is expressed onto the vaporizer element 1808, the user can trigger vaporization by inhaling through the inhalation outlet 1809. When the user inhales, a corresponding change in pressure is detected by the inhalation sensor. The signal is read by the control circuit 1803 which in turn activates the vaporizer element 1808. Fresh air may flow into the control device 1801 via an air inlet. After the air enters via the air inlet, air then flows through an upstream passageway in the housing 1802 and past the vaporizer element 1808 where the vapor is entrained into the air stream. The aerosol then continues through an outlet passageway and exits the housing 1802 via the inhalation outlet 1809. The inlet passageway and outlet passageway can be integral to the housing 1802 or cna be formed by the union of the housing 1802 and the cartridge housing 1811. The cavity formed by the union of the cartridge housing 1802 with the space immediately surrounding the vaporizer element 1808 may determine the particle or droplet size of the aerosol. A larger cavity size may allow for small droplets to accrete, resulting in an aerosol with a larger average particle size. The cavity size may be designed so as to produce the optimal particle size for the uptake of inhalation media. A secondary air inlet (not shown) may optionally be included at or downstream from the vaporizer element 1808 in order to provide additional air to mix with the aerosol and produce a cooler aerosol for inhalation by the user.

1810 may also contain a readable/writeable memory IC 1814, e.g. EEPROM, in order to store information including, but not limited to: the characteristics of the inhalation media, dosing information, control information, and usage information. The memory IC 1814 can be used to track how much inhalation media has been previously expressed and how much remains in the inhalation media storage area 1812. For example, a new cartridge 1810 may include a memory IC 1814 that has been programed with the number of optical encoder disk counts (limit) that corresponds to how far the plunger driver 1817 must advance to fully evacuate the inhalation media storage area 1812. Upon the dispensing of each dose, a running total of the number of counts that have been dispensed thus far can be written to the memory IC 1814 by the control circuit 1803. This is done via one or more electrical connectors. Before, during or after dispensing each dose, the control circuit 1803 can compare the running total of the number of counts that have been dispensed with the limit. When the numbers are equal, the control circuit 1803 can indicate that the cartridge 1810 is empty.

The embodiment shown in FIG. 18 employs a single cartridge 1810 and corresponding dispensing system, however, alternative embodiments exist that employ 2 or more cartridges 1810. In these embodiments, there can be a corresponding number of plunger drivers 1817 transmissions, and dispensing motors 1804. The control circuit 1803 can be configured to calculate the quantity of inhalation media to be dispensed from each cartridge 1810 onto the vaporizer element 1808 and drive the dispensing motors 1804 to achieve the desired dose. Such a multi-cartridge configuration can allow for on-demand blending of compositions created according to the disclosure with other compositions.

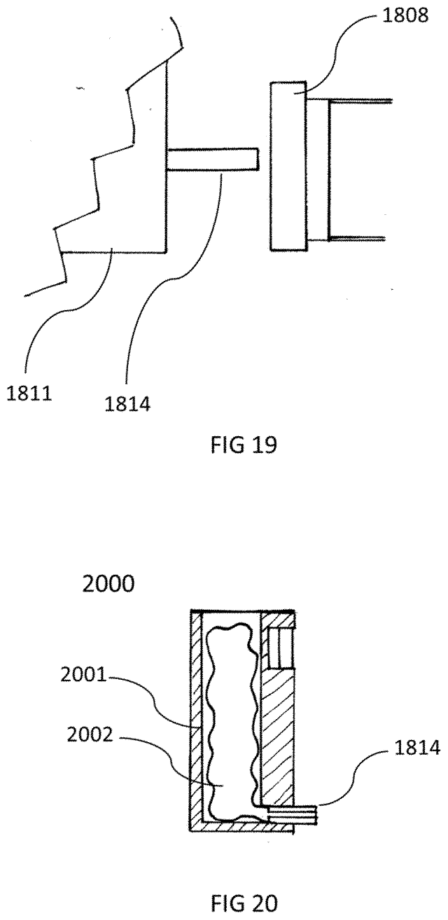

FIG. 19 shows a vaporizer element 1808 constructed according to an aspect of the disclosure. In this embodiment, the vaporizer element 1808 includes a heating element, substrate and heater leads. The substrate can be constructed using a thin piece of ceramic, glass, or other material that conducts thermal energy well while conducting electrical current poorly. Example dimensions of a substrate are 6 mm×6 mm×1 mm. By keeping the substrate thin, heat energy may more easily transfer from the heating element to the surface of the substrate that is closest to the cartridge outlet 1814. Keeping the substrate thin enables the use of materials, such as glass, that do not conduct thermal energy as rapidly as other suitable materials. The heating element is comprised of a resistive heating element that produces heat when electrical current passes through the material. The heating element can be a resistive compound that is deposited onto one or more surfaces of the substrate using deposition techniques commonly employed in the manufacture of electronic componentry. Alternatively, the heating element can be a resistance wire formed into a flat shape and brought into contact with one or more surfaces of the substrate or embedded within substrate. Typical resistance wire materials include alloys of nickel-chromium, titanium, Kanthal and other suitable materials. Typical resistance values for the heating element 801 range from 0.1 Ohms to 5 Ohms. The substrate is located in close proximity to the cartridge outlet 1814, preferably without touching. By maintaining proximity, small amounts or droplets of inhalation media can bridge the distance between the cartridge outlet 1814 and the substrate then spread out over the surface of the substrate. In addition, to enabling droplets to bridge, the condition of proximity without touching ensures that the cartridge outlet 1814 neither damages the substrate nor thermally couples with it, ensuring that the system does not waste energy heating the cartridge outlet 1814 thus limiting the potential degradation of inhalation media remaining in the cartridge outlet 1814. A typical distance between the substrate and cartridge outlet 1814 ranges from 0.1 mm to 1.0 mm. Alternative embodiments exist where the cartridge outlet 1814 is allowed to contact the substrate.

FIG. 20 shows a section view of an alternative embodiment of the cartridge 2000. In this embodiment, the cartridge housing 2001 has an internal portion that contains a flexible inhalation media bag 2002 which is connected to the cartridge outlet 1814. Inhalation media comprised of compositions according to the disclosure is stored in the inhalation media bag 2002. In this embodiment, the plunger driver 1807 presses directly onto the inhalation media bag 2002 in order to express inhalation media via the cartridge outlet 1814. The material(s) of the inhalation media bag 1814 are selected so as to minimize possible chemical reaction with the inhalation media.

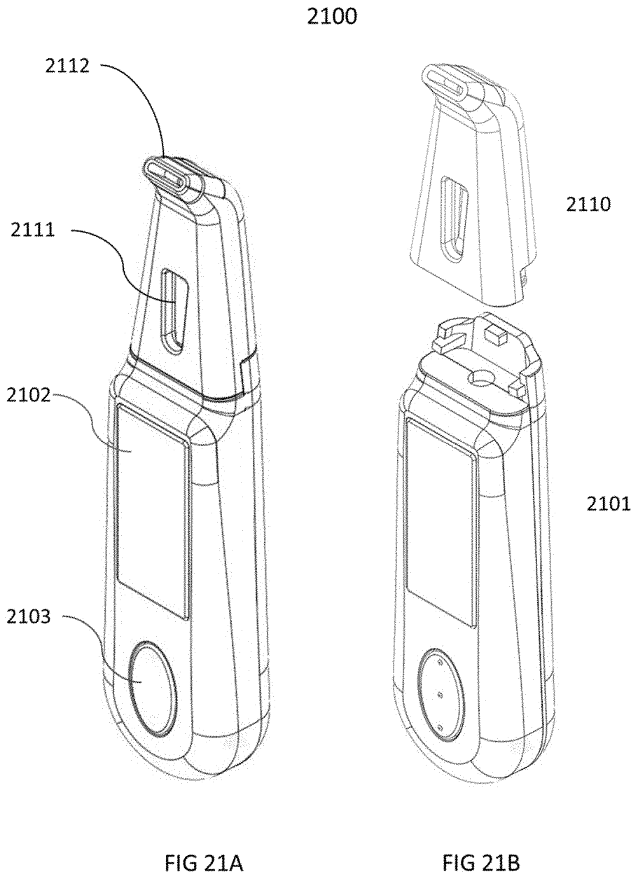

FIGS. 21A and 21B show an in-line constituent vaporizer article 2100 according to an aspect of the disclosure. The in-line constituent vaporizer article 2100 is comprised of in-line constituent control device 2101 and in-line constituent cartridge 2110. The in-line constituent control device 2101 can contain a screen 2102 and control pad 2103. The in-line constituent control device 2101 is substantially similar to control device 1801 with a primary difference being that the vaporizer element 1808 is not contained within in-line constituent control device 2101. The vaporizer element 1808 can be contained within in-line constituent cartridge 2110, which is substantially similar to cartridge 1810. The in-line constituent cartridge 2110 can have a viewing window 2111 and a mouthpiece 2112. The in-line constituent cartridge 2110 is connectably removable from in-line constituent control device 2101. The in-line constituent control device 2101 can have a plunger portal through which the plunger driver 1807 may extend. One or more guide features can be provided to assist with the mating of in-line constituent cartridge 2110 with in-line constituent control device 2101. A retention latch can also be provided to assist with retention. A retention latch can be comprised of a magnet which acts upon a piece of ferrous material or magnet located within in-line constituent cartridge 2110. A retention latch 3504 can alternatively be a mechanical feature shaped so as to provide a slight mechanical interference.

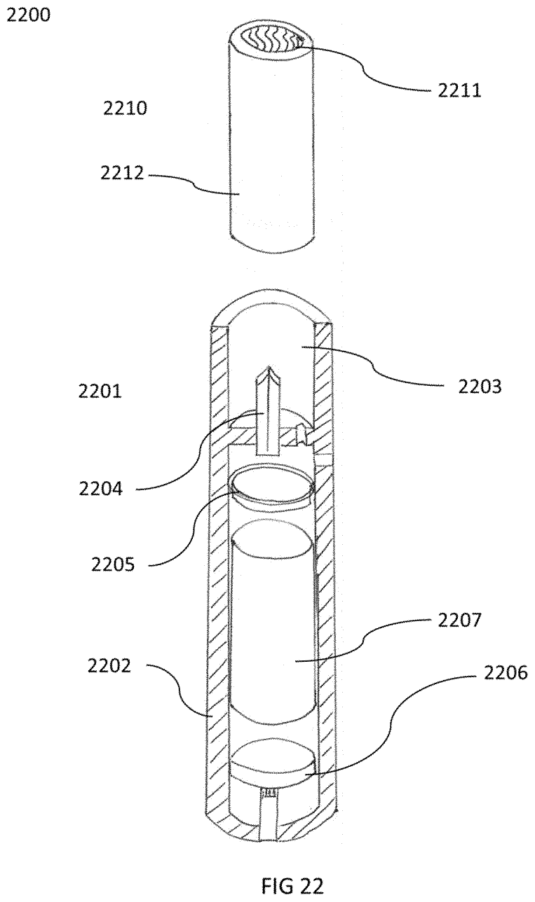

A certain class of vaporizers operates by heating a solid media, such as dried tobacco, herb, hemp or cannabis plant matter, including, but not limited to: Phillip Morris International IQOS, British American Tobacco Glo, Omura Series 1, PAX 2 and PAX 3. In such vaporizers, the solid media is heated to a temperature below the temperature of combustion in order to produce an aerosol without producing combustion and its associated byproducts. The solid media can be heated to or near the pyrolytic temperature of the material. The solid media may be pre-processed in order to promote aerosol formation. For example, the solid media can be flattened, dried, made into granules, mixed with binder material, and/or mixed with aerosol forming substances such as propylene glycol or glycerin. Importantly, compositions according to the present disclosure can be mixed into or sprayed onto solid media in order to provide enhanced organoleptic properties. FIG. 22 describes solid media vaporizer article 2200. The solid media vaporizer article 2200 is comprised of a solid media control device 2201 and solid media stick 2201. The solid media stick 2201 can be similar to a traditional combustible cigarette in form and composition; it can contain a solid media such as ground tobacco leaf which may be treated with compositions according to the present disclosure and a filter (not shown) wrapped inside a wrapper 2212. The filter can be situated between the solid media 2211 and the user's mouth in order to prevent particulate matter from being inhaled. The wrapper 2212 may be made from cigarette paper in order to give it the feel of a combustible cigarette. The wrapper 2212 can alternatively be made of plastic, metal, wood, or other material suitable for containing the solid media 2211.

The distal end of the solid media stick 2210 can be inserted into the heating cavity 2203 of the solid media control device 2201 such that the solid media heating element 2204 contacts the solid media 2211. When the solid media stick 2210 is properly inserted into the solid media control device 2201, solid media 2211 may be substantially disposed within the heating cavity 2203. When the user inhales on the proximal end of the solid media stick 2210, air enters the solid media control device 2201 through an air inlet located in the wall of solid media control device housing 2202. Air then flows through an air sensor opening located in air flow sensor 2205 which generates a signal which can be read by the solid media control device circuit 2206 which in turn can be configured to send power from the battery 2207 to the heating element 2204. When the heating element 2204 is energized, the solid media 2211 is heated to the point where an aerosol may be formed. The aerosol may be mixed and/or entrained in the air flow which passes through the filter then into the user's mouth. A secondary air inlet (not shown) can optionally be included at the heating cavity 2203 in order to provide additional air to mix with the aerosol and produce a cooler aerosol for inhalation by the user. In other embodiments, heater element 2204 activation may be provided by other means such as an air pressure sensor or button rather than an air flow sensor 2205.

Because the solid media vaporizer article 2200 heats a bulk amount of solid media 2211 rather than the positively placing of a pre-determined volume of inhalation media onto a heater for vaporization, the dosing precision offered by vaporizer article 1800 may not be possible. However, one way to provide for dose control is to have each solid media stick 2210 provide one dosing unit. Solid media sticks 2210 may be sold in pre-determined dosing levels, for example, in the case of cannabis containing solid media dosing sticks 2210, the sticks may be offered in THC levels of 2.5 mg, 5 mg, 7.5 mg and 10 mg. In the case of hemp containing solid media dosing sticks, for example, the sticks may be offered with CBD levels of 5 mg, 10 mg, 15 mg and 20 mg. In both examples, the user may use one or more sticks in combination in order to consume the desired dose.

Some users may prefer using solid media sticks 2210 instead of cartridges 1810 because solid media 2211 tends to retain more of the chemicals responsible for flavor, smell, physical and mental effects than the fluid isolates and distillates typically used in cartridges 1810. However, many of those chemicals may be found deep within the materials used to form the solid media 2211 and thus may not readily enter the aerosol stream. For clarity, this may be due to physical distance between the heating element 2204 and certain portions of the solid media 2211 resulting in less heating of certain portions of the solid media 2211. This may alternatively be due to the fact that certain chemicals may be “locked” within the materials used to form the solid media 2211 rather than sitting on the surface of those materials where they can be more readily volatilized by the heating element 2204. One way to increase the presence of these chemicals in the aerosol stream is to pre-treat the materials with a solvent then allow the materials to dry out before forming the solid media stick 2210. The treatment with solvent allows the chemicals to be “unlocked” from within the materials while the drying process tends to pull the chemicals toward the surfaces of the material as the solvent evaporates. This leaves the surfaces of the materials rich with the chemicals where they can be more easily volatilized. A number of different solvents may be appropriate for use in pre-treatment. Ethanol may be an especially good solvent for use with cannabis and hemp plant materials. In addition, plant materials may be sprayed with or soaked in compositions subjected to thermal processing according to the present disclosure so that the composition resides on the surfaces of the plant materials for easy volatilization.

An alternative to using plant material as the solid media 2211 has also been developed. Porous beads or porous strips of either organic (e.g., but not limited to cellulosic materials) or inorganic material (e.g., but not limited to metal oxides and ceramic) can be loaded with thermally treated compositions and used in place of the cannabis or hemp material in solid media sticks 2210.

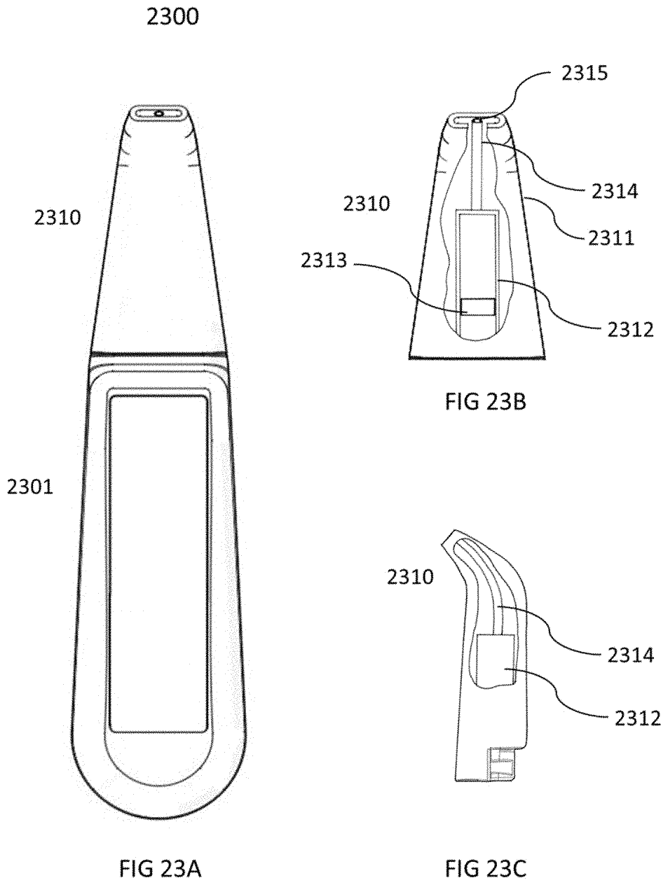

FIGS. 23A, 23B and 23C shows an ingestible media delivery article 2300 according to an aspect of the disclosure. Ingestible media delivery article 2300 is substantially similar to in-line constituent vaporizer article 2100 except that the vaporizer element 1808 is eliminated from in-line constituent cartridge 2110 to create an ingestible cartridge 2310. In this embodiment, the media inside the ingestible cartridge 2310 can be ingested orally and either swallowed by the user or placed under the tongue to be absorbed through sublingual tissue rather than inhaled. The media stored in media storage area 2312 can contain thermally treated compositions according to the present disclosure. The media stored in media storage area 2312 (shown in section view in FIG. 23B and solid view in FIG. 23C) can be expressed directly into the user's mouth via tube 2314 when the ingestible plunger 2313 is moved. The media, which can be in liquid or powder form, exits the tube 2314 via tube opening 2315. In one embodiment, ingestible control device 2301 can be identical to in-line constituent control device 2101. Through the ability to read memory IC 1814, the in-line constituent control device 2101 may differentiate between an in-line constituent cartridge 2110 and an ingestible cartridge 2310 and control each accordingly. This means that all the functionality offered by in-line constituent control device 2101 including, but not limited to: dose control, dispensing, data recording and sharing, dose compensation, and connectivity may also be delivered in an ingestible application; only the functions specifically related to generating an aerosol would not be applicable.

FIG. 24 shows an in-line constituent vaporizer article 2100 that contains a display screen 2102 configured to display the composition of in-line constituent cartridge 2110. Display screen 2102 can be configured to display a header that provides a general description and/or name of the inhalation media contained in in-line constituent cartridge 2110. Display screen 2102 can be further configured to display one or more constituents or characteristics 2404a-2404n of the inhalation media. Constituents can include, but are not limited to, cannabinoids, flavonoids, terpenes, terpenoids, drugs, medicines, active ingredients, preservatives, solvents, and carrier materials. Display screen 2102 can be further configured to display additional characteristics of the inhalation media including organoleptic properties, flavor and aroma information. Control pad 2103 can be used to scroll through the display in order to see additional constituents not displayed on the display screen 2102. Control pad 2103 can additionally be configured to select constituents for the purpose of indication to the in-line constituent vaporizer article 2100 which constituent the user desires to dose as well as the amount of the constituent the user wants to consume.

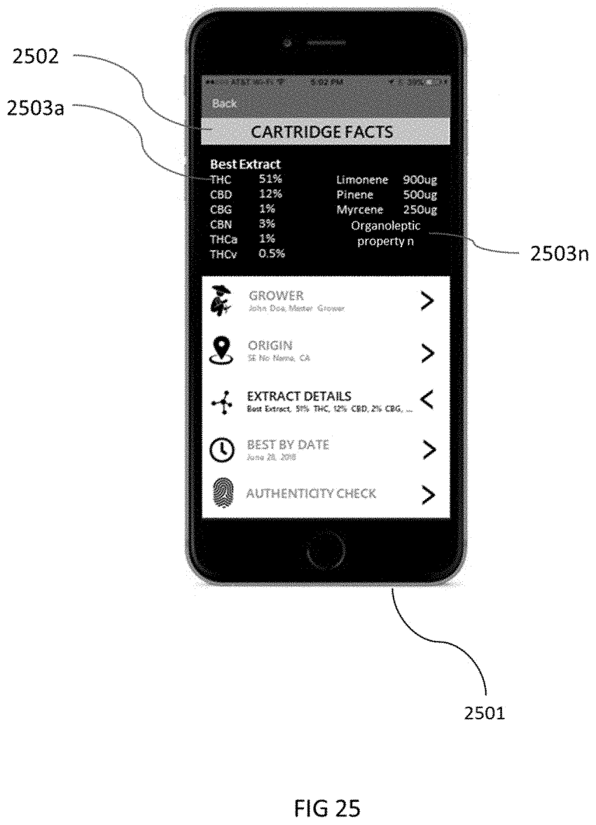

FIG. 25 shows a dose visualization application 2502 configured to communicate with in-line constituent vaporizer article 2100 and display the composition and characteristics of in-line constituent cartridge 2110. Dose visualization application 2502 can be configured to run on computing device 2501. Dose visualization application 2502 can be configured to display an application header and a constituent listing 2503a-2503n. Constituents can include, but are not limited to, cannabinoids, flavonoids, terpenes, terpenoids, drugs, medicines, active ingredients, preservatives, solvents, and carrier materials. Dose visualization application 2502 can be further configured to display additional characteristics of the inhalation media including organoleptic properties, flavor and aroma information. The user can use the controls provided by computing device 2501 to scroll through the constituent listing. Dose visualization application 2502 may be additionally configured to allow the user to select the constituent the user desires to dose as well as the amount of the constituent the user wants to consume in a dose. Dose visualization application can be additionally configured to display other information related to the inhalation media, including, but not limited to: the date of manufacture, expiration date, origin information, producer information, production process information, testing information, potency, and proof of authenticity.

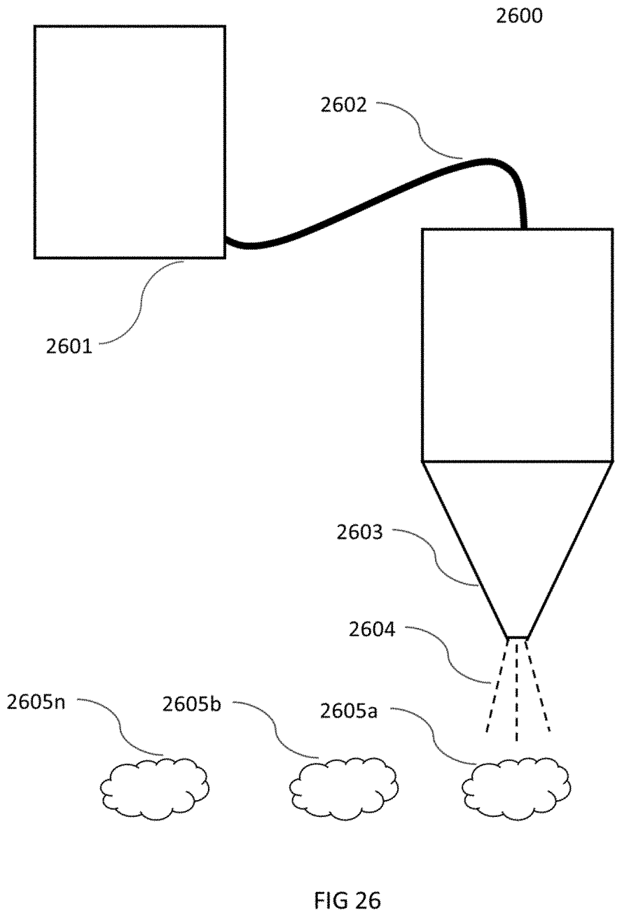

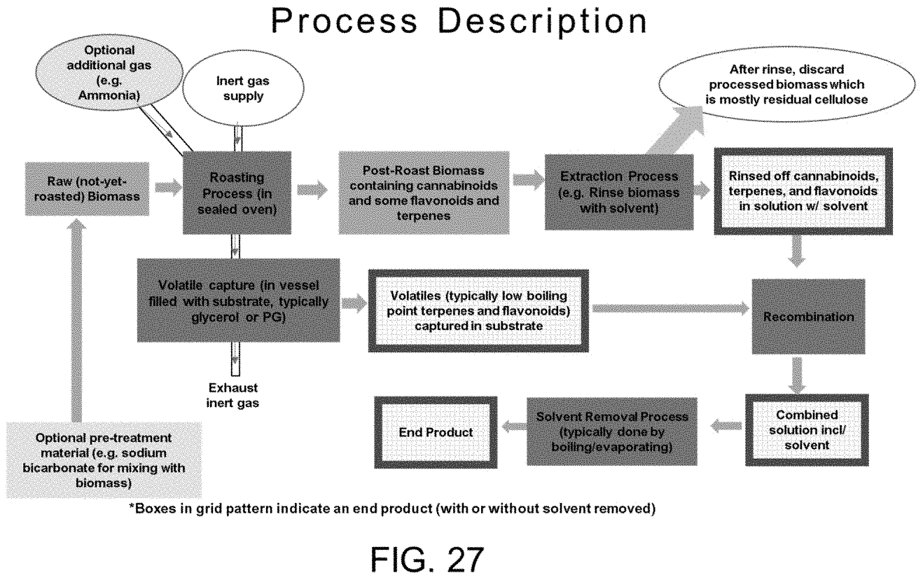

Certain consumable substrates can benefit from the application of thermally treated compositions constructed according to the present disclosure. For example, food items can be treated with such compositions in order to impart unique and/or superior organoleptic properties to the food items. Example food items include, but are not limited to; nuts, grains, flour, pastries, cookies, potato or other vegetable chips, meat snacks, popcorn and beverages, including, but not limited to; alcoholic drink bitters, coffee, tea, drink additive shots, energy shots, and beer. Capsules may also be filled with thermally treated compositions and used as nutritional supplements or medicines. By way of additional example, thermally treated compositions can also be made into soluble powder for the purpose of adding to water to create mixtures. Liposome encapsulants can be used to enable this. Food and supplement items need not be limited to human consumption and can also include animal food and supplement items. By way of additional example, inferior grade cannabis plant material can be treated with thermally treated compositions in order to impart superior organoleptic properties and thus improve its salability. Such treated cannabis plant material can then be smoked or vaporized by the user and deliver an experience more similar to that of a superior grade cannabis plant material. FIG. 26 shows an example spray treatment process 2600 and associated equipment for treating consumable substrates 2605 with thermally treated compositions. Thermally treated composition is stored in reservoir 2601 and connected to dispensing nozzle 2603 via pipe 2602. A spray of thermally treated composition 2604 is ejected from dispensing nozzle 2603 and onto consumable substrate material 2605a-2605n which can be configured in a production environment to pass by dispensing nozzle 2603. For certain substrates, including, but not limited to; beverages, tinctures, cosmetics, and topicals, it can be more appropriate to dispense thermally treated composition via a liquid stream rather than spray.

The following is a non-limiting example and embodiment of compositions having particular utility with respect to desirable organoleptic properties and chemical characteristics.

Furthermore, analytical methods previously described herein were used to determine the chemical characteristics of the composition according to the disclosure and the control composition. It is understood that the analytical methods described are general methods and represent techniques known to those skilled in the art.

The GC-MS spectra shown in FIG. 28 illustrates a general comparison between the processed composition embodied by the disclosure herein (Spectrum B) and the control composition (Spectrum A) created from equivalent starting materials and mass. The peaks between about 2.5 minutes and about 3.1 minutes are characteristic of ethanol contained in the compositions. The relative integrated area of each peak is within about 10 to about 15% of each other indicating the ethanol content of the control sample and composition according to the disclosure are essentially the same. Furthermore, the general cannabinoid content of the compositions, as indicated by signal peaks between about 4.5 minute and about 5.5 minute retention times, have essentially the same relative integrated areas. The fact the cannabinoid content is essentially the same, according to this analysis, is surprising and unexpected given the volatile nature, oxidative, and thermal degradation propensity of cannabinoids in general when exposed to temperatures above 75° C. That is to say, the composition according to the disclosure retain similar cannabinoid content to a composition made according to representative industry practices yet the composition according to the disclosure are subjected to much greater thermal conditions. Furthermore, the ability to thermally modify, alter, or augment chemical entities known for desirable flavor characteristics and further retain those describable chemical entities in proportion to retained cannabinoids in a single composition are enabled by the disclosure and further underscore the utility. This feature is further illustrated in the GC-MS spectrum of the processed composition according to the disclosure (Spectrum B) as compared to the control composition (Spectrum B) at elution times between about 6 minutes and about 7 minutes, denoted as Region A in FIG. 28. Specifically, as a non-limiting example, Spectrum B has contains signal peaks at about 6.5 minutes and 6.6 minutes, identified as 1a and 1b respectively, whereas Spectrum A does not. These peaks are primarily associated with esters of linoleic acid and their isomers which are known to have desirable organoleptic properties. Table 3 illustrates general chemical class of fatty acid esters as non-limiting examples, also known in part as Omega 3 and Omega 6 fatty acids.

TABLE 3
Non-Limiting Examples of Modified Linolenic
Acid According to the Disclosure

| | Example Name |
|---|---|
| Peak 1a | |
| 1 | 2(1H)-Naphthalenone, octahydro-4a-methyl-7-(methylethyl)-,(4a |
| | alpha.,7beta, 8a.beta.) |
| 2 | n-propyl 9,12-octadecadienoate |
| 3 | 12,15 octadecadienoic acid, methyl ester |
| 4 | 6,9 Octadecadienoic acid, methyl ester |
| 5 | linoleic acid ethyl ester |
| 6 | butyl 9,12-octadecadienoate |
| 7 | 9,12-ocatdecadienoyl chloride (z,z) |
| 8 | Methyl 9-cis, 11 trans-octadecadienoate |
| 9 | 9,12 octadecadienoic acid (z,z) hydroxy-1-(hydroxymethyl)ethyl |
| | ester |
| 10 | 11,14-octadecadienoic acid methyl ester |
| Peak 1b | |
| 11 | 9,12,15- Octadecatrieoic acid ethyl ester (Z,Z,Z) |
| 12 | methyl 8,11,14 heptadecatrienoate |
| 13 | Methyl 2, hydroxy-octadeca 9,12,15 trienoate |
| 14 | 7,10,13, Hexadecatrienolic acid methyl ester |
| 15 | Butyl 9.12,15 octadecatrienoate |
| 16 | n propyl 9,12,15 octadecatrienoate |

In addition, a detailed evaluation of volatile flavonoid and general terpenoid class compounds form GC-MS analysis of compositions according to the disclosure further embody desirable organoleptic features. Specifically, the ability to retain desirable flavonoids, terpenoids, sesquiterpenoids, esters, alkanes, alkenes, and other known volatile chemical compositions having the general formula of, but not limited to, CxHyOz, or CxHySz, or CxHyNz, where in X is 5 to 26, and Y is 4 to 44, and Z is 1 to 6, or any combination thereof, at conditions described according to the disclosure, while simultaneously retaining cannabinoids, such as but not limited to, cannabigerol (CBG), a cannabichromene (CBC), a cannabidiol (CBD), tetrahydrocannabinol (THC), cannabinol (CBN), cannabielsoin (CBE), iso-tetrahydrocannabinol (iso-THC), cannabicyclol (CBL), cannabicitran (CBT), CBG-type cannabinoid a CBC-type cannabinoid, a CBD-type cannabinoid, a THC-type cannabinoid, a CBN-type cannabinoid, a CBE-type cannabinoid, an iso-THC-type cannabinoid, a CBL-type cannabinoid, a CBT-type cannabinoid, a CBG-type cannabinoid, or combinations thereof in a single composition were unexpected and previously not known. Table 4 illustrates the general class of terpenoids that exemplify desirable organoleptic properties contained in a single composition according to the disclosure

TABLE 4
Generalized classes of terpenoids and flavonoids found in compositions
according to the disclosure as non-limiting examples.

Oxetane, 2,2-dimethyl
Pentanal
Disulfide, dimethyl
Pyridine
Ethanone, 1-(1-cyclohexen-1-yl)- -
Acetamide
3-Furaldehyde
1,3,8-p-Menthatriene -
p-Mentha-1,5,8-triene -
5-Hepten-2-one, 6-methyl- -
p-Mentha-1,5,8-triene -
2,6-Heptadien-1-ol, 2,4-dimethyl- -
Benzaldehyde -
Butyrolactone -
Bicyclo[3.1.1]heptane, 6,6-dimethyl-2-methylene-, (1S)- -
p-(1-Propenyl)-toluene -
Benzene, 1-methyl-4-(1-methylethenyl)--
p-Mentha-1,5,8-triene -
1,7,7-Trimethylbicyclo[2.2.1]heptan-2-ol -
Ethanone, 1-(4-methylphenyl)- -
Caryophyllene -
Humulene -
4a,8-Dimethyl-2-(prop-1-en-2-yl)-1,2,3,4,4a,5,6,7-octahydronaphthalene -
Naphthalene, decahydro-4a-methyl-1-methylene-7-(1-methylethylidene)-,
(4aR-trans)- -
(4aR,8aS)-4a-Methyl-1-methylene-7-(propan-2-ylidene)
decahydronaphthalene -
alpha.-Guaiene -
1,2-Benzenediol, O-(4-butylbenzoyl)-O′-(isobutoxycarbonyl)- -
Naphthalene, decahydro-4a-methyl-1-methylene-7-(1-methylethylidene)-,
(4aR-trans)- -
Neophytadiene -
Azulene, 1,2,3,3a,4,5,6,7-octahydro-1,4-dimethyl-7-(1-methylethenyl)-,
[1R-(1.alpha.,3a.beta.,4.alpha.,7.beta.)]- -
Hydroxy-.delta. 9-tetrahydrocannabinol, 8-.alpha. -
Allyl acetate -
(1R)-2,6,6-Trimethylbicyclo[3.1.1]hept-2-ene -
Bicyclo[3.1.1]heptane, 6,6-dimethyl-2-methylene-,(1S)- -
1H-4-Oxabenzo(f)cyclobut(cd)inden-8-ol,
1a-.alpha.,2,3,3a,8b-.alpha.,8c-.alpha.-hexahydro-1,1,3atrimethyl-

Surprisingly, the control formulation did not exhibit any such compounds. The formation of the desirable compounds further underscores the utility of the disclosure and, as such, imparts favorable characteristics to cannabinoid-containing compositions. Furthermore the combination of such compounds in conjunction with the retention of the cannabinoids in a single composition further differentiate compositions according to the disclosure from those previously known in the art.

### Methods & Materials

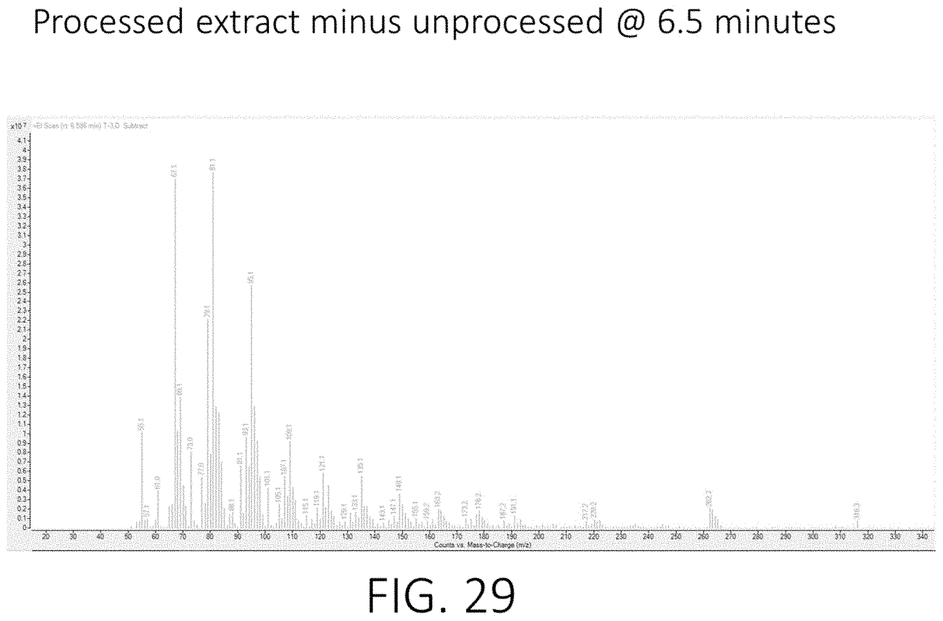

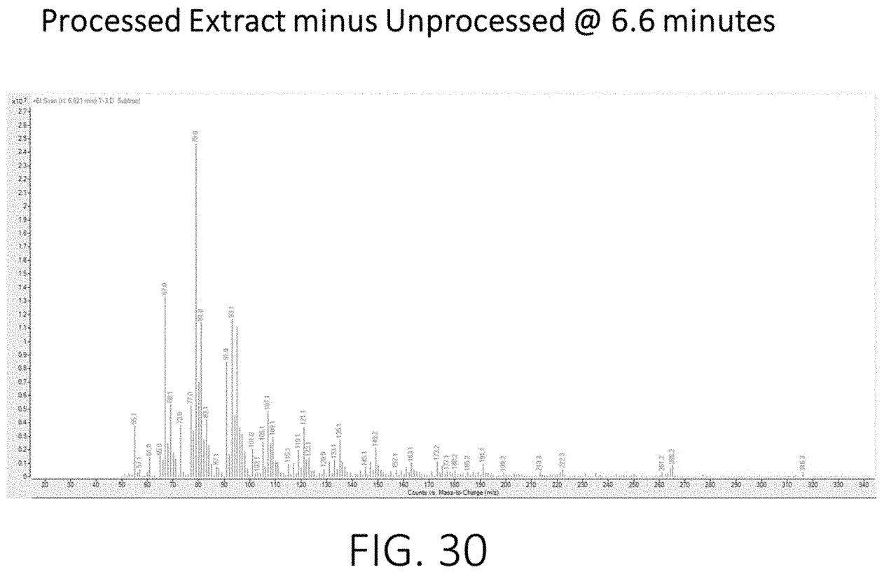

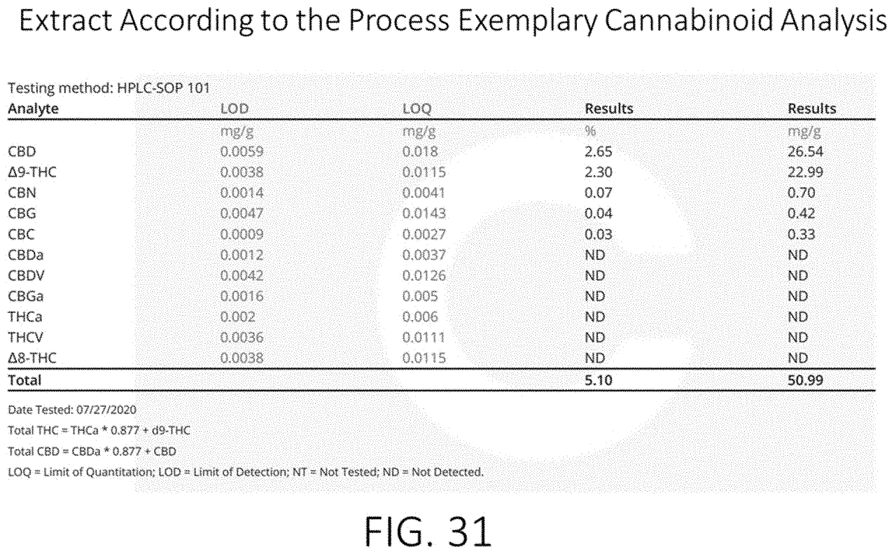

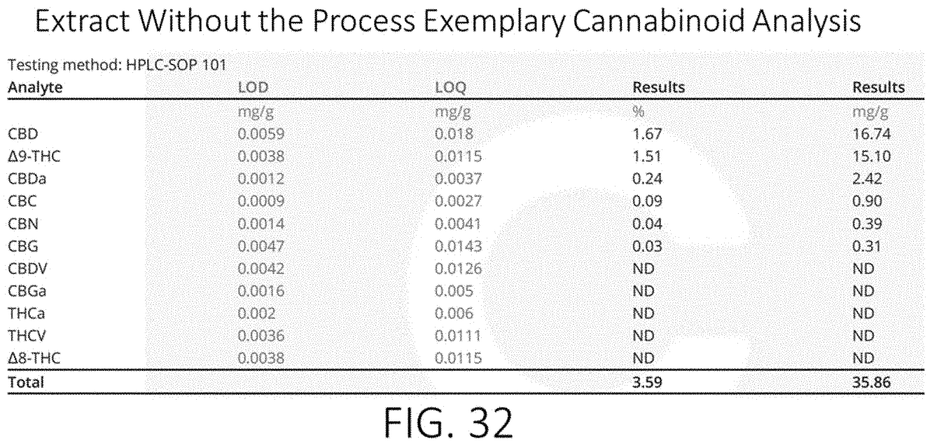

GC-Scan: FIGS. 28-30

Samples were diluted with ACN and passed through a 0.22 um filter into an amber glass vial. The sample was injected onto an Agilent intuvo 9000 with an Agilent 7000D GC/MS Triple Quad. Inlet temperature was ramped from 180 C to 280 C at 300 C/min with a constant flow of He at 1.4 ml/min. Chromatographic separation was achieved using an Agilent 122-5512UI-INT, DB-5 MS UI, 30 m×250 um×0.25 um at an initial temperature of 120 C held for one minute. The column temperature is then increased at a rate of 25 C/min to 300 C and is held at 300 C for 0.55 min. Data was collected using electron ionization with a source temperature of 280 C and the detector in an MS2 scan mode. Mass spectra were extracted from the chromatogram at the peak maximum and compared with the NIST library of similar mass spectra.

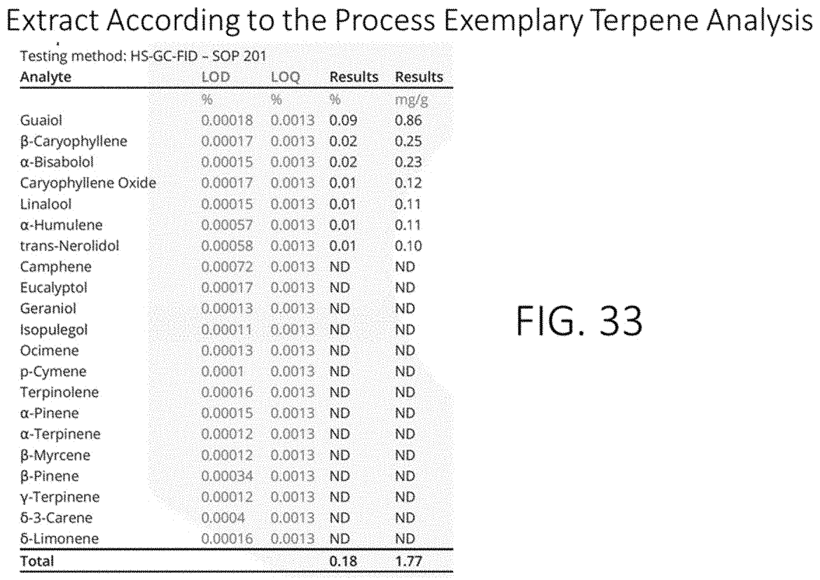

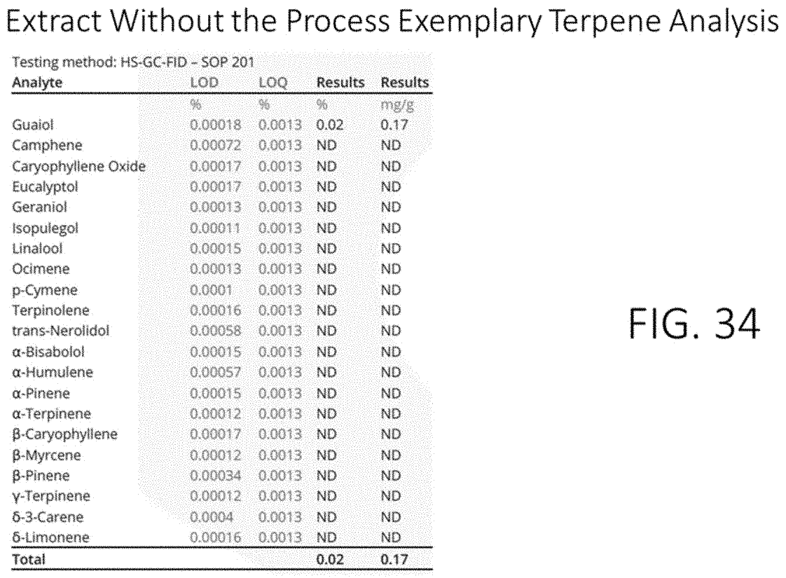

Terps: FIGS. 33-34

Samples are extracted with DMA and measured using full evaporative headspace analysis using an Agilent 7697A headspace sampler, Agilent Intuvo GC Oven, and Agilent Flame Ionization Detector. 20 ul of the extract was added to a 20 ml headspace vial and heated at 120 C for 10 min. One ul of headspace is transferred to a GC via a loop and transfer line heated to 120 C and 140 C, respectively. Inlet temperature was held constant at 200 C with a constant flow of He at 150 ml/min and a split ratio of 50:1. Chromatographic separation was achieved using an Agilent DB-35 MS UI, 30 m×250 um×0.25 um, at an initial temperature of 60 C held for 0.3 min. The column temperature is then increased at a rate of 55 C/min to 150 C, increased at a rate of 35 C/min to 250 C, and is held at 250 C for 0.5 min. Data was collected using flame ionization detector with a heater temperature of 300 C in constant makeup (N2) & fuel (H2) flow.

While the disclosure has been described in terms of exemplary embodiments, those skilled in the art will recognize that the disclosure can be practiced with modifications in the spirit and scope of the appended claims. These examples given above are merely illustrative and are not meant to be an exhaustive list of all possible designs, embodiments, applications, or modifications of the disclosure.

1. A cannabis composition, comprising at least one cannabinoid and at least one flavonoid.

2. The composition of claim 1, wherein the at least one flavonoid is a bioflavonoid, an isoflavonoid, a neoflavonoid, or combinations thereof.

3.-6. (canceled)

7. The composition of 1, wherein the composition further comprises at least one terpenoid and/or terpene.

8.-9. (canceled)

10. The composition of claim 1, wherein the composition is derived or extracted from a plant material.

11.-15. (canceled)

16. The composition of claim 1, wherein the composition is derived by exposing the plant substrate to heat under a gas atmosphere.

17. The composition of claim 16, wherein the plant substrate is heated to a temperature of from about 100° C. to about 500° C.

18. (canceled)

19. The composition of claim 16, wherein the gas environment comprises at least one of nitrogen gas, argon gas, carbon dioxide gas, helium gas, or oxygen gas.

20.-22. (canceled)

23. The composition of claim 16, wherein the gas environment is essentially free of oxygen.

24.-25. (canceled)

26. The composition of claim 16, wherein the gas environment is at atmospheric pressure, below atmospheric pressure, or above atmospheric pressure.

27. The composition of claim 16, wherein the gas environment comprises a gas flow.

28.-30. (canceled)

31. The composition of claim 1, wherein the at least one cannabinoid comprises THC, CBD, CBC, CBN, CBG, CBL, a THC-type cannabinoid, a CBD-type cannabinoid, a CBC-type cannabinoid, a CBN-type cannabinoid, a CBG-type cannabinoid, a CBL-type cannabinoid, or combinations thereof.

32. The composition of claim 31, wherein the THC-type cannabinoid is tetrahydrocannabinolic acid (THCA), tetrahydrocannabivarin (THCV), Δ9-THC, Δ8-THC, 8-hydroxy-Δ9-tetrahydrocannabinol, or combinations thereof.

33.-34. (canceled)

35. The composition of claim 31, wherein the cannabinoid is CBD.

36. The composition of claim 1, wherein the composition further comprises a fatty acid or ester thereof.

37. The composition of claim 36, wherein the fatty acid or ester thereof comprises a short- or medium-chain fatty acid or ester thereof.

38. The composition of claim 36, wherein the fatty acid ester is a C1-C6 ester.

39. The composition of claim 36, wherein the fatty acid ester is a methyl ester.

40. The composition of claim 1, wherein the composition further comprises: 2,2-dimethyloxetane; pentanal; dimethyl disulfide; pyridine; 1-acetylcyclohexene; acetamide; 3-furaldehyde; p-mentha-1,3,8-triene; p-mentha-1,5,8-triene; 6-methyl-5-hepten-2-one; 2,4-dimethyl-2,6-heptadien-1-ol; benzaldehyde; butyrolactone; (1R,5S)-2-methylene-6,6-dimethylbicyclo[3.1.1]heptane; p-(1-propenyl)-toluene; p-cymenene; acetophenone; caryophyllene; humulene; 2-isopropenyl-4a,8-dimethyl-1,2,3,4,4a,5,6,7-octahydronaphthalene; alpha-guaiene; 1,2-benzenediol, [2-(2-methylpropoxycarbonyloxy)phenyl] 4-butylbenzoate; (8aR)-8a-methyl-4-methylidene-6-propan-2-ylidene-2,3,4a,5,7,8-hexahydro-1H-naphthalene; neophytadiene; 1,4-dimethyl-7-prop-1-en-2-yl-1,2,3,3a,4,5,6,7-octahydroazulene; cannabichromene; (6aR,8R,10aR)-6,6,9-trimethyl-3-pentyl-6a,7,8,10a-tetrahydrobenzo[c]chromene-1,8-diol; 1H-tetrazole; allyl acetate; (1R)-2,6,6-trimethylbicyclo[3.1.1]hept-2-ene; (1R,5S)-2-methylene-6,6-dimethylbicyclo[3.1.1]heptane; 2,3-Dihydroxypropyl acetate; (1aS,3aR,8bR,8cR)-1,1,3a-trimethyl-6-pentyl-1a,2,3,3a,8b,8c-hexahydro-1H-4-oxabenzo[f]cyclobuta[cd]inden-8-ol; dronabinol; 7-isopropyl-4a,8a-dimethyloctahydro-1(2H)-naphthalenone; n-propyl 9,12-octadecadienoate; methyl 9,12,15-octadecatrienoate; 6,9-octadecadienoic acid methyl ester; ethyl linolenate; butyl 9,12-octadecadienoate; 9,12-octadecadienoyl chloride (z,z); methyl 9-cis, 11 trans-octadecadienoate; 2-mono-linolein; methyl (11E,14E)-octadeca-11,14-dienoate; ethyl linolenate; methyl heptadeca-8,11,14-trienoate; methyl (9Z,12Z,15Z)-2-hydroxyoctadeca-9,12,15-trienoate; methyl (7E,10E,13E)-hexadeca-7,10,13-trienoate; butyl (9Z,12Z,15Z)-9,12,15-octadecatrienoate; methyl (5Z,8Z,11Z,14Z)-icosa-5,8,11,14-tetraenoate; n-propyl 9,12,15-octadecatrienoate, or combinations thereof.

41.-62. (canceled)

63. The composition of claim 1, wherein the inhalation delivery is by inhaler, nebulizer, vaporizer, aerosolizer, or a smoking device.

64. (canceled)

65. The composition of claim 63, wherein the smoking device is a cigarette or cigar comprising tobacco, hemp cannabis, herbs, spices, or combinations thereof.

66. The composition of claim 63, wherein the device is an electronic smoking device.

67.-69. (canceled)

70. The composition of claim 1, wherein the composition has at least two additional peaks with a retention time of about 4.5, about 4.6, about 4.7, about 4.8, about 4.9, or about 7.15 minutes in a GC-MS chromatogram.

71.-72. (canceled)

73. The composition of claim 1, wherein the composition is characterized by a GC-MS chromatogram of FIG. 28.

74. A process for producing the composition of claim 1, comprising:
(i) providing an unprocessed plant feedstock in a gas environment;
(ii) heating the unprocessed plant feedstock in the gas environment to afford a processed plant product and at least one volatile compound; and
(iii) extracting the treated plant product of (ii) with a first solvent or solvent system thereby providing a first extract.

75.-136. (canceled)
